SEQUENCE LISTING (16 sequences)

SEQ ID NO: 1 - protein, 1363 aa, Artificial Sequence
  Description of Artificial Sequence Synthetic polypeptide
MLSFVDTRILLLLAVTSYLATSQHVSEASAGRKGPRGDKGPQGERGPPGPPGRDGEDGPPGPPGPPGPPGLGGNFAAQYDPSKAADFGPGPMGLMGPRGPPGASGPPGPPGFQGVPGEPGEPGQTGPQGPRGPPGPPGKAGEDGHPGKPGRPGERGVAGPQGARGFPGTPGLPGFKGIRGHNGLDGQKGQPGTPGTKGEPGAPGENGTPGQPGARGLPGERGRIGAPGPAGARGSDGSAGPTGPAGPIGAAGPPGFPGAPGAKGEIGPAGNVGPTGPAGPRGEIGLPGSSGPVGPPGNPGANGLPGAKGATGLPGVAGAPGLPGPRGIPGPPGPAGPSGARGLVGEPGPAGAKGESGNKGEPGAAGPPGPPGPSGEEGKRGSNGEPGSAGPPGPAGLRGVPGSRGLPGADGRAGVMGPAGNRGASGPVGAKGPNGDAGRPGEPGLMGPRGLPGQPGSPGPAGKEGPVGFPGADGRVGPIGPAGNRGEPGNIGFPGPKGPTGEPGKPGEKGNVGLAGPRGAPGPEGNNGAQGPPGVTGNQGAKGETGPAGPPGFQGLPGPSGPAGEAGKPGERGLHGEFGVPGPAGPRGERGLPGESGAVGPAGPPGSRGPSGPPGPDGNKGEPGNVGPAGAPGPAGPGGIPGERGVAGVPGGKGEKGAPGLRGDTGATGRDGARGLPGAIGAPGPAGGAGDRGEGGPAGPAGPAGARGIPGERGEPGPVGPSGFAGPPGAAGQPGAKGERGPKGPKGETGPTGAIGPIGASGPPGPVGAAGPAGPRGDAGPPGMTGFPGAAGRVGPPGPAGITGPPGPPGPAGKDGPRGLRGDVGPVGRTGEQGIAGPPGFAGEKGPSGEAGAAGPPGTPGPQGILGAPGILGLPGSRGERGLPGIAGATGEPGPLGVSGPPGARGPSGPVGSPGPNGAPGEAGRDGNPGNDGPPGRDGAPGFKGERGAPGNPGPSGALGAPGPHGQVGPSGKPGNRGDPGPVGPVGPAGAFGPRGLAGPQGPRGEKGEPGDKGHRGLPGLKGHNGLQGLPGLAGQHGDQGPPGNNGPAGPRGPPGPSGPPGKDGRNGLPGPIGPAGVRGSHGSQGPAGPPGPPGPPGPPGPNGGGYEVGFDAEYYRADQPSLRPKDYEVDATLKTLNNQIETLLTPEGSKKNPARTCRDLRLSHPEWSSGFYWIDPNQGCTADAIRAYCDFATGETCIHASLEDIPTKTWYVSKNPKDKKHIWFGETINGGTQFEYNGEGVTTKDMATQLAFMRLLANHASQNITYHCKNSIAYMDEETGNLKKAVILQGSNDVELRAEGNSRFTFSVLVDGCSKKNNKWGKTIIEYRTNKPSRLPILDIAPLDIGGADQEFGLHIGPVCFK

SEQ ID NO: 2 - DNA, 4362 bp, Artificial Sequence
  Description of Artificial Sequence Synthetic polynucleotide
atgttcagcttcgtggacagcagactgctgctgctgatcgccgccaccgtgctgctgacaagaggccaaggcgaagaggacattcagaccggcagctgcgtgcaagacggtctgacttacaacgacaaggacgtgtggaagcccgagccctgtcagatctgcgtgtgcgacagcggtaacatcctctgcgacgaggtgatctgcgaggacacaagcgactgccccaacgccgagatccccttcggcgagtgctgtcccatctgccccgacgtagatgctagccccgtgtaccccgagtcggctggagtagagggtcctaagggcgacacgggaccccggggggatcgtggacttcccggcccacccggcagagacggaatacccggccaacccggtctacctggccctcctggccccccgggcccaccgggactgggggggaacttcgccccacagatgagctatgggtacgacgagaaaagtgctggagttgccgtccccggcccgatgggccccgccggcccacggggtctacccggcccgcccggcgcacccggcccgcaaggttttcaaggccctcccggcgagcccggcgaaccgggtgcgagtggtccaatgggaccgcgagggcccgccggcccacccggaaagaatggcgacgacggggaagccggcaagcctggccgtcccggggagaggggcccccccggcccccaaggcgctcgcggcctgcccggcacggccggtttgcccggcatgaagggccatagaggtttcagcggactggatggtgcaaaaggagaaccgggaccggcgggaccgaagggggaacccgggtcccccggcgagaatggagccccgggacagatgggcccccgaggtctccccggcgaacgaggacgccctggcccttctggtcccgccggtgctcgtgggaatgatggggcgcccggcgctgctggacctcccggtcctactggaccggccggcccaccgggctttcctggggccgccggtgcaaagggggaaaccggccctcaaggagcaaggggttccgagggtccccaaggtgcgaggggtgagcccggcccccccggccccgccggtgccgcgggaccggccgggaatcccggggcagacggtcaacctggcgccaaaggtgcaaccggagctccgggcatagctggtgctcccggcttccccggggcacgcggtgcgcccggccctcaaggaccaagcggcgctcctggtcctaagggaaatagtggagagcccggcgcgcccggcaataaaggcgataccggcgccaaaggggagcccggccccgccggagtgcaaggaccgccgggtcctgctggcgaagaaggcaaacgcggcgctcgtggtgaacccgggccggcgggtctgccggggccggctggtgagagaggagcgcccggcagcagaggctttcccggcgcagatggtattgcgggtccaaaaggacctcccggtgagaggggttctcccggcgccgtcggtccgaaaggaagtcctggtgaggctggaaggcccggtgaacccggcttgcccggcgctaaaggtttgaccggctcgcccggctcacctggacctgatggtaaaaccggccccccgggtcccgccggacaagacggtcggcccggcccgcctgggcctcctggggcaaggggccaagccggggttatgggatttcctgggccaaagggcgcagccggcgaacctgggaagcccggtgagagaggtgctcccggcccgcccggcgcagtgggagctgccggcaaagacggtgaggccggagcgcaaggcccaccgggaccaaccgggcctgccggcgagcgcggtgaacaaggcccggccggtgctcccggatttcaaggcctgcccggccctgcgggcccccccggcgaggcgggcaaacctggggaacaaggcgttccgggagacgccggtgcccccgggccggctggagcgaggggtgaacgtggctttccgggggaacgaggtgtgcaaggccctcccggcccccaaggcccccgtggcgccaatggagcccccggcaatgatggggctaaaggcgacgctggggcgcccggagctcccggcaaccaaggaccgcctggattacaagggatgcctggggaacgcggcgccgccggccttcccggcgcgaaaggcgataggggcgaccccggacccaagggtgccgacggggctcccggcaaggatggcctacgggggctgactgggccaattgggccccctggcccggccggcgcacccggcgacaagggtgaagccggcccgagcggccctccgggccccaccggcgcccgaggagctcccggcgatcgtggcgaacccggccctcccggccccgccggatttgctggcccgccgggtgcagacggccaacctggcgctaagggcgaaaccggcgacgcgggcgcaaaaggcgatgctggcccacccggccctgccggacctacgggggctcccggccctgctggggcggtgggtgctcctgggccgaagggagcgagaggctcagccgggccccccggcgcgacgggtttccctggagcggccggccgtgtcgggccccccggtccttctggtaatataggcttacccggcccgcccggtccaagtggaaaggaggggggtaaagggccgagaggcgagactggccccgcggggaggcccggcgagccgggacccgctgggccacctggtccccctggcgaaaagggcagccccggcgccgacgggcccataggagcccccggcacccctggtccccaaggtatagccggacagaggggggtggtcggactccccggtcaacgcggtgagagaggatttccgggcctgcccgggccaagcggagaacccggaaaacaaggtccgagcggctcgccgggggagcgtggtcctcctggaccaatgggtccgcctggtcttgccggcccccccggagaagcgggcagagagggagcaccgggggccgagggcgcccccggccgtgacggcgcagccgggcctaagggcgatcgaggggaaaccggccccgccggaccacccggcgcgcccggcgcgcccggtgcccctgggcccgttgggcctgccgggaaaaacggtgaccggggcgaaaccgggccggccgggcctgcgggaccacctgggcctgctggagcacgaggacccgccggaccacaaggcccgcgcggtgataaaggtgagaccggcgagcaaggcgacagaggcatgaaaggccatagaggctttagcggcctacaaggcccgcccggccctcccggcgccccgggtgaacaagggccctctggcgcatctggacctgccggcccacgaggcccgcctggcagcgccggggcagctggaaaggacggtcttaatggcttgcctgggccaataggtccacccggtccaagaggtaggaccggcgaggtaggtcctgtcggtccgcccgggccgcccggtccccccggcccgcctggccccccctccggcggtttcgacttcagcttcctgccacagcccccccaagagaaggcccacgacggcggcagatactacagagccgacgacgccaacgtgatgagagacagagacctggaggtggacaccaccctgaagagcctgtctcagcagatcgagaacatcagaagccccgagggcacaagaaagaaccccgctagaacctgcagagacctgaagatgtgccacggcgactggaagagcggcgagtactggatcgaccccaaccaaggctgcaacctggacgccatcaaggtgtactgcaacatggagacgggagagacatgcgtgtaccccacccaagccaccatcgctcagaagaactggtatctgagcaagaaccccaaggagaagaagcacgtgtggttcggcgagaccatgagcgacggctttcagttcgagtacggcggcgagggcagcaaccccgccgacgtggccattcagctgaccttcctgagactgatgagcaccgaggccacacagaacgtgacctaccactgcaagaacagcgtggcctacatggaccacgacaccggcaacctgaagaaggccctgctgctgcaaggcgccaacgagatcgagatcagagccgagggcaacagcagattcacctacggcgtgaccgaggacggctgcacaagccacaccggcgcctggggcaagaccgtgatcgagtacaagaccaccaagacaagcagactgcccatcatcgacctggcccccatggacgtgggtgctcctgatcaagagttcggcatcgacatcggccccgtgtgcttcctgtga

SEQ ID NO: 3 - protein, 1453 aa, Artificial Sequence
  Description of Artificial Sequence Synthetic polypeptide
MFSFVDSRLLLLIAATVLLTRGQGEEDIQTGSCVQDGLTYNDKDVWKPEPCQICVCDSGNILCDEVICEDTSDCPNAEIPFGECCPICPDVDASPVYPESAGVEGPKGDTGPRGDRGLPGPPGRDGIPGQPGLPGPPGPPGPPGLGGNFAPQMSYGYDEKSAGVAVPGPMGPAGPRGLPGPPGAPGPQGFQGPPGEPGEPGASGPMGPRGPAGPPGKNGDDGEAGKPGRPGERGPPGPQGARGLPGTAGLPGMKGHRGFSGLDGAKGEPGPAGPKGEPGSPGENGAPGQMGPRGLPGERGRPGPSGPAGARGNDGAPGAAGPPGPTGPAGPPGFPGAAGAKGETGPQGARGSEGPQGARGEPGPPGPAGAAGPAGNPGADGQPGAKGATGAPGIAGAPGFPGARGAPGPQGPSGAPGPKGNSGEPGAPGNKGDTGAKGEPGPAGVQGPPGPAGEEGKRGARGEPGPAGLPGPAGERGAPGSRGFPGADGIAGPKGPPGERGSPGAVGPKGSPGEAGRPGEPGLPGAKGLTGSPGSPGPDGKTGPPGPAGQDGRPGPPGPPGARGQAGVMGFPGPKGAAGEPGKPGERGAPGPPGAVGAAGKDGEAGAQGPPGPTGPAGERGEQGPAGAPGFQGLPGPAGPPGEAGKPGEQGVPGDAGAPGPAGARGERGFPGERGVQGPPGPQGPRGANGAPGNDGAKGDAGAPGAPGNQGPPGLQGMPGERGAAGLPGAKGDRGDPGPKGADGAPGKDGLRGLTGPIGPPGPAGAPGDKGEAGPSGPPGPTGARGAPGDRGEPGPPGPAGFAGPPGADGQPGAKGETGDAGAKGDAGPPGPAGPTGAPGPAGAVGAPGPKGARGSAGPPGATGFPGAAGRVGPPGPSGNIGLPGPPGPSGKEGGKGPRGETGPAGRPGEPGPAGPPGPPGEKGSPGADGPIGAPGTPGPQGIAGQRGVVGLPGQRGERGFPGLPGPSGEPGKQGPSGSPGERGPPGPMGPPGLAGPPGEAGREGAPGAEGAPGRDGAAGPKGDRGETGPAGPPGAPGAPGAPGPVGPAGKNGDRGETGPAGPAGPPGPAGARGPAGPQGPRGDKGETGEQGDRGMKGHRGFSGLQGPPGPPGAPGEQGPSGASGPAGPRGPPGSAGAAGKDGLNGLPGPIGPPGPRGRTGEVGPVGPPGPPGPPGPPGPPSGGFDFSFLPQPPQEKAHDGGRYYRADDANVMRDRDLEVDTTLKSLSQQIENIRSPEGTRKNPARTCRDLKMCHGDWKSGEYWIDPNQGCNLDAIKVYCNMETGETCVYPTQATIAQKNWYLSKNPKEKKHVWFGETMSDGFQFEYGGEGSNPADVAIQLTFLRLMSTEATQNVTYHCKNSVAYMDHDTGNLKKALLLQGANEIEIRAEGNSRFTYGVTEDGCTSHTGAWGKTVIEYKTTKTSRLPIIDLAPMDVGAPDQEFGIDIGPVCFL

SEQ ID NO: 4 - DNA, 4092 bp, Artificial Sequence
  Description of Artificial Sequence Synthetic polynucleotide
atgctgagcttcgtggacacaagaatcctgctgctgctggccgtgacaagctacctggcgacaagccaacacgtgagcgaggcaagcgccggtagaaaagggccgcgtggggacaagggcccgcaaggtgagcgaggaccccccggcccccccggtcgtgatggtgaagacggacctcccggcccacctggaccccccggccctcctgggctgggaggcaattttgccgctcagtacgatccaagcaaggcagccgactttgggcctggtccaatgggcctcatgggtccgagaggtccaccgggtgcgagcgggcctcccggaccgcctggttttcaaggcgttccgggagagcccggtgagcccggccaaactggcccgcaagggccaaggggaccacccggcccgcctggtaaggctggggaagacggtcatccgggcaagccggggaggcctggagaacggggtgtagctgggccgcaaggcgcgaggggctttcctggtacgcccgggttacccgggtttaaaggcattagagggcacaacggcctggacggacagaagggacaacccggtactcccggcacaaagggcgaacccggcgctcccggtgaaaatggcacccctggacaacccggggcgagaggactccccggcgagaggggacgaatcggtgcgcccggccccgccggcgcaaggggctccgatgggagtgccggccccactggtccggccggccccattggggccgccggcccgcccggctttccgggggcacccggtgctaagggggaaataggacccgcggggaacgtaggcccgaccggccccgctggacctagaggggaaatcggcctgcccgggtcaagcggccctgtcgggccccccggcaatcccggcgcgaatggcttacccggcgcgaaaggagccactgggttgcctggcgtggccggcgcacccggattgcccggaccccgggggattcccggcccccccgggccggccggcccatcgggtgctaggggactagtaggggagcccggccccgccggcgcgaagggcgagagcggcaataaaggagaacccggcgccgctggtccaccgggaccgcctggaccgtctggggaggaagggaaaagagggagtaatggggagcccggcagcgctggccctcccggacctgccggcctccgaggagtacctggaagcagagggttgccgggcgcagatggcagagctggcgtgatgggtccggctggtaacagaggagcaagcggccccgttggtgccaagggcccgaacggtgatgccggccgacccggcgaacctggtctcatgggaccacgcggattgccggggcagcctggctcacctggcccggcgggaaaagaagggcctgtgggatttcctggagcggatggccgggttggtcctatcggaccggccggaaataggggagaacccggtaacatcgggttcccgggccccaagggtccgacgggtgagccgggtaagcccggcgagaaaggtaatgtgggattagctggaccaagaggggcaccgggccccgaaggcaacaatggcgcgcaaggcccgcccggggttacgggcaaccaaggggctaagggcgaaacgggacccgccggtcctcctggattccaaggacttccggggccgagcgggcccgccggcgaagccggcaaacccggcgaacgtggcctgcatggcgagttcggcgtgcccgggcccgccgggccgcgtggcgagcggggactacctggtgagagcggtgcggttggacccgccgggccgcccggctctcgagggccttcgggcccgcccggcccggatggtaacaagggtgaacctggcaatgtgggccccgccggcgctccgggacccgccggacccgggggcatacctggcgaacggggtgtggccggcgtgcctggaggcaagggcgaaaagggggctccggggctgagaggggataccggcgctaccggccgggacggagcccgggggttacccggcgcgatcggagcccccggcccggccggcggtgccggcgacagaggagagggcggccccgccggtcccgccggccccgccggtgcgcgaggtatacctggtgagaggggcgaacctggacctgttggcccaagcgggtttgctgggccgcccggtgccgcgggacaacccggcgccaagggagagcgagggcccaagggaccaaagggtgagactggccctacgggggctataggcccgattggtgcgagtggcccccctggccctgtgggcgccgcgggtccggccgggcctcggggagatgctgggccacccggaatgactggtttcccgggtgcagccggccgtgtcggtccaccgggccccgccggcataactggcccgcccggtccgccggggccggccggcaaggacggaccaagaggtcttcgtggtgacgtggggcccgtgggaagaaccggcgagcaaggcattgcgggtccccctggctttgccggcgaaaaaggcccgtcgggtgaagcgggggcagctggaccccccggaactcctggcccccaaggcatacttggagcgcccggcatacttggccttcccggctcaagaggcgaacgagggctacctggcatcgctggggccactggggaacccggaccacttggcgtgagtggccctcccggtgcgagggggccaagtggtcccgttggttctcccggccctaatggagcacctggggaagccggccgtgacggtaatcctggcaatgatggaccacccggccgtgatggtgcacctgggttcaagggagaaagaggagcccccggcaaccccggtccgtccggcgccttaggagcacccggcccacacgggcaagtggggccaagtgggaagccggggaatcggggcgaccccggaccggtagggcccgtgggtcccgccggcgccttcggtcctcgagggctagccgggccacaaggcccacgcggcgaaaagggcgagcccggcgacaagggccacagaggcttgcccgggttaaaaggtcacaacggactgcaaggcctccccggtctggcgggacagcatggtgaccaaggcccgccggggaacaacggccccgcgggcccgcgtggccctccggggccctcgggtccgcctgggaaggatggacggaacggcttgcccggaccgataggaccggccggggtccgtggcagccacggcagccaaggccccgccggccctcccggcccgcccggccctcccggcccacctggcccaaatgggggaggctacgaggtgggcttcgacgccgagtactacagagccgatcagccaagcctgagacccaaggactacgaggtggacgccaccctgaagaccctgaacaatcagatcgagaccctgctgacccccgagggtagcaaaaagaacccggctagaacctgcagagacctgagactgagccaccccgagtggagcagcggcttctactggatcgaccccaaccaaggctgcaccgccgacgccatcagagcctactgcgacttcgccaccggcgagacctgcatccacgctagcctggaggacatccccaccaagacctggtacgtgagcaagaaccccaaggacaagaagcacatctggttcggcgagaccatcaacggcggcacacagttcgagtacaacggcgagggcgtgaccaccaaggacatggccacacagctggccttcatgagactgctggccaaccacgcttctcagaacatcacctaccactgcaagaacagcatcgcctacatggacgaggagaccggcaacctgaagaaggccgtgatcctgcaaggcagcaacgacgtggagctgagagctgaggggaattcccgtttcaccttcagcgtgctggtggacggctgcagcaagaagaataacaagtggggcaagaccatcatcgagtacagaaccaacaagccaagcagactgcccatcctggacatcgcccccctggacatcggcggcgccgaccaagagttcggcctgcacatcggccccgtgtgcttcaagtga

SEQ ID NO: 5 - protein, 1985 aa, Artificial Sequence
  Description of Artificial Sequence Synthetic polypeptide
MSSDSEMAVFGEAAPYLRKPEKERTEAQNRPFDSKKACFAVDDKEVYVKGMIQNRENDKVTVKTLDDRTITLNTAQVFPMNPPKFDKIEDMAMMTHLHEPAVLYNLKERYAAWMIYTYSGLFCVTVNPYKWLPVYNPEVVMAYRGKKRQEAPPHIFSISDNAYQFMLTDRDNQSILITGESGAGKTVNTKRVIQYFATIAVPGDKKKEPQTGKMQGTLEDQIIQANPLLEAFGNAKTVRNDNSSRFGKFIRIHFGATGKLASADIETYLLEKSRVTFQLSSERSYHIFYQIMSNKKPELIDLLLISTNPFDFPFISQGEVTVASIDDSEELLATDNAIDILGFSSEEKVGIYKLTGAVMHYGNMKFKQKQREEQAEPDGTEVADKAGYLMGLNSAEMLKGLCCPRVKVGNEYVTKGQNVQQVTNSVGALAKAVYEKMFLWMVARINQQLDTKQARQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSANFQKPKPTKGKAEAHFSLVHYAGTVDYNIVGWLDKNKDPLNETVVGLYQKSSLKLLAFLFSNYAGAEAGDCGGSKKGGKKKGSSFQTVSAVFRENLNKLMTNLRSTHPHFVRCLIPNETKTPGVMDHYLVIHQLRCNGVLEGIRICRKGFPSRILYGDFRQRYRILNASAIPEGQFIDSKNASEKLLNSINVDREQYRFGHTKVFFKAGLLGLLEEMRDEKLATLMTRTQAVCRGYLMRVEFKKMMERRDSILCIQDNIRSFMNVKHWPWMNLFFKIQPLLKSAEAEKEMATMKENFKRAQEELARSEARRKELEEKMVSLLQEKNDLQLQVQSETENLMDAEERCEGLIKSKIQLEAKVKELNEKLEEEEEINSELVAKKRNLEDKCFSLKKDIDDLELTLTKVEKEKHATENKVKNLSEEMTALEENISKLTKEKKSLQEAHQQTLDGLQVEEDKVNGLIKINAKLEQQTDDLEGCLEQEKKLRADLGRLKRKLEGDLKISQESITDLENAQQQMEEKLKKKEFEISQLQTKIDGEQVHSLQLQKKIKELQARTEELEEDIEAEHTLRAKLERQRSDLARELEEISERLEEASGATSAQVELNKKREAEFQKMRRDLEEATLQHEATAATLRKKHADSVAELGEQIDNLQRVKQKLQKEKSELKMEMDDMASNIETITKSKSNMERMCRTMEDQFNELKAKDDQQTQLIHDLNMQKARLQTQNGELSHQVEEKESLISQLSKGKQALTQQLEELKRQLEEETKAKNALAHALQSSRHDCDLLREQYEEEQEGKAEVQRALSKANSEVAQWRTKYETDAIQRTEELEEAKKKLAQRLQEAEENTEAVNSKCASLEKTKQRLQGEVDDLMLDLERANTTCATLGKKQRNFDKILADWKQKLDESHAELEAAQKESRSLSTEIFKMKNAYEEVVDQLETLRRENKNLQEEIHDVTEQIAETGKNLQEMEKTKKQVEQEKSDLQVALEEVEGSLEHEESKILRVQLELSQLKSELDRKVAEKDEEMEQLKRNCQRAAEATQSLLEAESRSRKDALRLKKKMEGDLNEMEVHLGHANRQVAETQKHLRTVQGQLKGSQLHLGDALRSDEALKERLAIVGRRNGLLVEELEETKVALEQAERSRRLSEQELQDASDRVQLLLSRCNTSLINIKKKLETNIAQCQAEVENSVQESRNAEEKAKKAITDAAMMAEELKKEQDTSAHLERMKKNLEQTVKDMQHRLDEAEQLALKGDKKQIQKLENRVRELESELDTEQKRGAEALKGAHKYERKVKEMAYQAEEDHKNSLRLQDLVDKLQAKVNAYKRQAEEAEEQANTQLSRCRRVQHELEEAEERVDIAESQVNKLRAKSRDVGSQVRADTWAWLLLKESRDPGAQRSHRESLEAAHWSLLLVMPSQMPPTQQRLL

SEQ ID NO: 6 - DNA, 5958 bp, Artificial Sequence
  Description of Artificial Sequence Synthetic polynucleotide
atgagcagcgacagcgagatggccgtgttcggcgaggccgccccctacctgagaaagcccgaaaaagaaagaacggaggctcagaacagacccttcgacagcaaaaaggcctgcttcgccgtggacgacaaagaggtgtacgtgaagggcatgattcagaacagagagaacgacaaggtgaccgtgaaaacccttgacgatcgtaccatcaccctgaacaccgcccaagtgttccccatgaacccccccaagttcgataagatcgaagacatggccatgatgacccacctgcacgagcccgccgtgctgtacaacctgaaggaaagatacgctgcctggatgatctacacctacagcggcctgttctgcgtgaccgtgaacccctacaagtggctgcccgtgtacaaccccgaggtggtgatggcctacagaggcaagaagagacaagaggcccccccccacatctttagcattagcgacaacgcctatcagttcatgctgaccgacagagacaatcagagcatcctgatcaccggcgagagcggcgccggcaagaccgtgaacaccaagagagtgattcagtacttcgccaccatcgccgtgcccggcgacaaaaaaaaagaaccgcaaacgggcaagatgcaaggcaccctggaggatcagatcatccaagccaaccccctactggaggccttcggcaacgccaagaccgtgagaaacgacaacagcagcagattcggcaagttcatcagaatccacttcggcgccaccggcaagctggctagcgccgacatcgagacctacctgctggagaagagcagagtgaccttccaactaagctccgagagaagctaccacatcttctatcagatcatgagcaacaagaagcccgaactgatcgatttactgctgatcagcaccaaccccttcgacttccccttcatcagccaaggcgaagtaaccgtggctagcatcgacgacagcgaggagctgctggccaccgacaacgccatcgacatcctgggcttcagcagcgaggagaaggtgggcatctacaagctgaccggcgccgtgatgcactacggcaacatgaagttcaagcaaaaacagagagaggagcaagctgagcctgatggcaccgaggtggccgacaaggccggctacctgatgggtctgaacagcgccgagatgctgaagggcctgtgctgcccaagagtgaaggtgggcaacgagtacgtgaccaagggacagaacgtgcagcaagtgaccaacagcgtgggcgccctggccaaggccgtgtacgagaagatgttcctgtggatggtggctagaatcaatcagcaactggacaccaagcaagctagacagtacttcatcggcgtgctggatatagccggcttcgagatcttcgacttcaactcactggagcagctttgcatcaacttcaccaatgagaagttgcagcaattcttcaaccaccacatgttcgtgctggaacaagaggaatataagaaggagggcatcgagtgggagttcatcgacttcggcatggacctggccgcctgcatcgagctgatcgaaaaacccatgggcatcttctcaattctggaagaagagtgtatgttccccaaggccaccgacacaagcttcaagaacaagctgtacgatcagcacctgggcaagagcgccaactttcagaagcccaagcccaccaagggcaaggccgaagcccacttcagcctggtgcactacgccggcaccgtggactacaacatcgtgggctggctggacaagaacaaggaccccctgaacgagaccgtggtgggcctgtatcagaagagcagcctgaagctgctggccttcctgttcagcaactacgccggcgcggaagcgggggattgcggcggcagcaagaagggcggcaagaagaagggcagcagctttcagaccgtgagcgccgtgttcagagagaacttaaataagctgatgaccaacctgagaagcacccacccccacttcgtgagatgcctgatccccaacgagaccaagacccccggcgtgatggaccactacctggtgatccatcagctgagatgcaacggtgttctggagggtatcagaatctgcagaaagggcttcccaagcagaatcctgtacggcgacttcagacagagatacagaatcctgaacgctagcgccatccccgagggacagttcattgatagcaagaacgcaagcgagaagctgctgaacagcatcaacgtggacagagagcagtatagattcggccacaccaaggtgttcttcaaggccggcctgctgggcctgctagaggagatgagagacgagaaattggccacgctgatgacaagaacccaagccgtgtgcagaggttatctgatgcgcgtggagttcaagaaaatgatggagagaagagacagcatcctgtgcatccaagacaacatcagaagcttcatgaacgtgaagcactggccctggatgaacctgttcttcaagattcagcccctgctgaagtcagcggaggcggaaaaggaaatggccaccatgaaggagaacttcaagagagcccaagaggagctggctagaagcgaggctagaagaaaggagctcgaggagaagatggtgagcctgctgcaagagaagaacgacctgcagctgcaagtccaaagcgagaccgagaacctgatggatgcagaggagcgatgtgagggtctgattaagtccaaaattcagctggaagccaaagtgaaggaactgaatgagaagctagaagaggaggaagaaataaacagcgagctggtggccaagaagcgcaacctggaggacaagtgcttcagcctgaagaaggacatcgacgatcttgagctgaccctgaccaaggtggagaaggagaagcacgccaccgagaacaaggtgaagaacctgagcgaggagatgaccgccctggaggaaaacattagcaagctgaccaaagagaagaagtcgctgcaagaggctcaccaacagactctggatggcctgcaagtggaggaggataaagtaaacggcttgatcaagattaacgcaaagttggaacaacagaccgacgatctggagggctgcttagaacaagaaaagaagctgagagccgacctgggcagactgaagagaaagctggagggcgacctgaagataagccaagagagcatcaccgacctggagaacgctcagcagcagatggaggagaagctgaagaagaaagaatttgagatcagccaactgcagaccaagatcgacggcgagcaagtgcacagccttcagttacaaaagaagatcaaggagctacaagctagaaccgaggagcttgaggaagacatcgaagccgaacacaccctgagagcgaaactggagagacagagaagcgacctggctagagagctggaggaaatcagcgagagactcgaggaggctagcggcgccacttccgcccaagtggagctgaacaagaagagagaggccgagtttcagaagatgagaagagacctggaagaggccactctgcagcacgaggccaccgccgccaccctgagaaagaagcacgccgacagcgtggccgagctgggcgagcagatcgacaacctgcagagagtgaagcaaaaattacaaaaagaaaagagtgagctgaaaatggagatggacgacatggctagcaacatcgagaccatcaccaagagcaagagcaacatggagagaatgtgcagaaccatggaggatcagttcaacgagctgaaggccaaggacgatcagcagacacagctgatccacgacctgaacatgcagaaggctagactgcagacacagaacggcgagctgagccaccaagtggaggagaaggaaagcctgatttctcagctgagcaagggcaagcaagccctgacacagcaattagaggaactgaagaggcagctcgaggaggagaccaaggccaaaaacgccctggcccacgccctgcagagcagcagacacgactgcgacctgctgcgggagcagtacgaggaagagcaagagggtaaggctgaggtgcagcgagccctgagcaaggccaacagcgaggtggctcagtggagaaccaagtacgagaccgacgccattcagagaactgaagaattggaggaagcgaagaagaagctcgcccaacgattgcaagaagccgaagagaacaccgaggccgtgaacagcaagtgcgctagcctggagaagacgaagcaaagactacaaggtgaggtggatgatctgatgctggacctggagagagccaacaccacctgcgccaccctgggcaagaagcagagaaacttcgacaaaatcctggctgactggaagcagaagctggacgagagccacgccgagctggaagccgctcagaaggaaagcagatccttaagcaccgagatcttcaagatgaagaacgcctacgaagaggtggtcgatcagctggagaccctgagaagagagaacaagaaccttcaagaggagatccacgacgtgaccgagcagatcgccgagaccggtaaaaacctacaagagatggagaagaccaagaagcaagtggaacaagagaagtccgatctgcaagtggccctggaagaagtagagggcagccttgaacatgaggagtcgaaaatcctgagagtacagctggagctgtctcagctgaagtctgagttggatagaaaagtcgctgaaaaggacgaggagatggagcagctgaagagaaactgtcagagagccgccgaggccacacagagcctgttagaggccgaatcgagaagcagaaaagacgcactgagactgaagaagaagatggaaggcgatttgaacgagatggaggtgcacctgggccacgccaacagacaagtggccgaaacacagaagcacctgagaaccgtgcaaggacagctgaagggctctcagctgcaccttggcgacgccctaagaagcgatgaagcactgaaggaacgtttagctatcgtgggcagaagaaacggcctgctggttgaggagcttgaagagacgaaagtagcccttgagcaagccgaaagaagcagaagactgagcgagcaagagctacaagacgctagcgaccgtgtacagctgttgctgagccgttgtaacacaagcctgatcaacatcaagaagaaactggagacaaacatcgctcagtgccaagccgaggtggagaacagcgtgcaagagagcagaaatgccgaggagaaggccaagaaggccatcaccgacgccgccatgatggccgaggagctgaaaaaagagcaagacacgtctgctcatctggagagaatgaagaagaacctggagcagaccgtgaaggacatgcagcacagactggacgaggccgaacagcttgccctgaagggagacaaaaaacaaattcagaagctggagaaccgagtgcgcgaactggagagcgaactggataccgagcagaagagaggcgccgaggcactcaaaggggcccacaagtacgagagaaaagttaaggagatggcctaccaagccgaagaggatcacaagaacagcctgaggctgcaagacctagtggacaagctgcaagccaaagtgaacgcctacaagagacaagccgaggaagcagaagaacaagccaacacacagctgtctaggtgcagaagagtgcagcacgagctggaggaggctgaagagagagtcgacatagcggagagccaagtgaataaattaagagcgaagagccgggacgtgggcagccaagtgagagccgacacctgggcctggctgctgctgaaagagagcagagaccccggcgctcagagaagccacagagagagcttagaggcagcgcactggagcctgctgctggtgatgccatctcagatgccccccacacagcagcgcctgctttga

SEQ ID NO: 7 - protein, 1453 aa, Artificial Sequence; identical to SEQ ID NO: 3
  Description of Artificial Sequence Synthetic polypeptide

SEQ ID NO: 8 - protein, 1453 aa, Gallus gallus
MFSFVDSRLLLLIAATVLLTRGQGEEDIQTGSCVQDGLTYNDKDVWKPEPCQICVCDSGNILCDEVICEDTSDCPNAEIPFGECCPICPDVDASPVYPESAGVEGPKGDTGPRGDRGLPGPPGRDGIPGQPGLPGPPGPPGPPGLGGNFAPQMSYGYDEKSAGVAVPGPMGPAGPRGLPGPPGAPGPQGFQGPPGEPGEPGASGPMGPRGPAGPPGKNGDDGEAGKPGRPGERGPPGPQGARGLPGTAGLPGMKGHRGFSGLDGAKGEPGPAGPKGEPGSPGENGAPGQMGPRGLPGERGRPGPSGPAGARGNDGAPGAAGPPGPTGPAGPPGFPGAAGAKGETGPQGARGSEGPQGARGEPGPPGPAGAAGPAGNPGADGQPGAKGATGAPGIAGAPGFPGARPSPGPQGPSGAPGPKGNSGEPGAPGNKGDTGAKGEPGPAGVQGPPGPAGEEGKRGARGEPGPAGLPGPAGERGAPGSRGFPGADGIAGPKGPPGERGSPGAVGPKGSPGEAGRPGEPGLPGAKGLTGSPGSPGPDGKTGPPGPAGQDGRPGPPGPPGARGQAGVMGFPGPKGAAGEPGKPGERGAPGPPGAVGAAGKDGEAGAQGPPGPTGPAGERGEQGPAGAPGFQGLPGPAGPPGEAGKPGEQGVPGDAGAPGPAGARGERGFPGERGVQGPPGPQGPRGANGAPGNDGAKGDAGAPGAPGNQGPPGLQGMPGERGAAGLPGAKGDRGDPGPKGADGAPGKDGLRGLTGPIGPPGPAGAPGDKGEAGPPGPAGPTGARGAPGDRGEPGPPGPAGFAGPPGADGQPGAKGETGDAGAKGDAGPPGPAGPTGAPGPAGAVGAPGPKGARGSAGPPGATGFPGAAGRVGPPGPSGNIGLPGPPGPSGKEGGKGPRGETGPAGRPGEPGPAGPPGPPGEKGSPGADGPIGAPGTPGPQGIAGQRGVVGLPGQRGERGFPGLPGPSGEPGKQGPSGSPGERGPPGPMGPPGLAGPPGEAGREGAPGAEGAPGRDGAAGPKGDRGETGPAGPPGAPGAPGAPGPVGPAGKNGDRGETGPAGPAGPPGPAGARGPAGPQGPRGDKGETGEQGDRGMKGHRGFSGLQGPPGPPGAPGEQGPSGASGPAGPRGPPGSAGAAGKDGLNGLPGPIGPPGPRGRTGEVGPVGPPGPPGPPGPPGPPSGGFDFSFLPQPPQEKAHDGGRYYRADDANVMRDRDLEVDTTLKSLSQQIENIRSPEGTRKNPARTCRDLKMCHGDWKSGEYWIDPNQGCNLDAIKVYCNMETGETCVYPTQATIAQKNWYLSKNPKEKKHVWFGETMSDGFQFEYGGEGSNPADVAIQLTFLRLMSTEATQNVTYHCKNSVAYMDHDTGNLKKALLLQGANEIEIRAEGNSRFTYGVTEDGCTSHTGAWGKTVIEYKTTKTSRLPIIDLAPMDVGAPDQEFGIDIGPVCFL

SEQ ID NO: 9 - protein, 15 aa, Tyrannosaurus rex
GAPGPQGPSGAPGPK

SEQ ID NO: 10 - protein, 33 aa, Brachylophosaurus canadensis
GLTGPIGPPGPAGAPGDKGEAGPSGPPGPTGAR

SEQ ID NO: 11 - protein, 1363 aa, Gallus gallus
MLSFVDTRILLLLAVTSYLATSQHVSEASAGRKGPRGDKGPQGERGPPGPPGRDGEDGPPGPPGPPGPPGLGGNFAAQYDPSKAADFGPGPMGLMGPRGPPGASGPPGPPGFQGVPGEPGEPGQTGPQGPRGPPGPPGKAGEDGHPGKPGRPGERGVAGPQGARGFPGTPGLPGFKGIRGHNGLDGQKGQPGTPGTKGEPGAPGENGTPGQPGARGLPGERGRIGAPGPAGARGSDGSAGPTGPAGPIGAAGPPGFPGAPGAKGEIGPAGNVGPTGPAGPRGEIGLPGSSGPVGPPGNPGANGLPGAKGAAGLPGVAGAPGLPGPRGIPGPPGPAGPSGARGLVGEPGPAGAKGESGNKGEPGAAGPPGPPGPSGEEGKRGSNGEPGSAGPPGPAGLRGVPGSRGLPGADGRAGVMGPAGNRGASGPVGAKGPNGDAGRPGEPGLMGPRGLPGQPGSPGPAGKEGPVGFPGADGRVGPIGPAGNRGEPGNIGFPGPKGPTGEPGKPGEKGNVGLAGPRGAPGPEGNNGAQGPPGVTGNQGAKGETGPAGPPGFQGLPGPSGPAGEAGKPGERGLHGEFGVPGPAGPRGERGLPGESGAVGPAGPIGSRGPSGPPGPDGNKGEPGNVGPAGAPGPAGPGGIPGERGVAGVPGGKGEKGAPGLRGDTGATGRDGARGLPGAIGAPGPAGGAGDRGEGGPAGPAGPAGARGIPGERGEPGPVGPSGFAGPPGAAGQPGAKGERGPKGPKGETGPTGAIGPIGASGPPGPVGAAGPAGPRGDAGPPGMTGFPGAAGRVGPPGPAGITGPPGPPGPAGKDGPRGLRGDVGPVGRTGEQGIAGPPGFAGEKGPSGEAGAAGPPGTPGPQGILGAPGILGLPGSRGERGLPGIAGATGEPGPLGVSGPPGARGPSGPVGSPGPNGAPGEAGRDGNPGNDGPPGRDGAPGFKGERGAPGNPGPSGALGAPGPHGQVGPSGKPGNRGDPGPVGPVGPAGAFGPRGLAGPQGPRGEKGEPGDKGHRGLPGLKGHNGLQGLPGLAGQHGDQGPPGNNGPAGPRGPPGPSGPPGKDGRNGLPGPIGPAGVRGSHGSQGPAGPPGPPGPPGPPGPNGGGYEVGFDAEYYRADQPSLRPKDYEVDATLKTLNNQIETLLTPEGSKKNPARTCRDLRLSHPEWSSGFYWIDPNQGCTADAIRAYCDFATGETCIHASLEDIPTKTWYVSKNPKDKKHIWFGETINGGTQFEYNGEGVTTKDMATQLAFMRLLANHASQNITYHCKNSIAYMDEETGNLKKAVILQGSNDVELRAEGNSRFTFSVLVDGCSKKNNKWGKTIIEYRTNKPSRLPILDIAPLDIGGADQEFGLHIGPVCFK

SEQ ID NO: 12 - protein, 18 aa, Tyrannosaurus rex
GLPGESGAVGPAGPPGSR

SEQ ID NO: 13 - protein, 18 aa, Brachylophosaurus canadensis
GATGLPGVAGAPGLPGPR

SEQ ID NO: 14 - protein, 1985 aa, Artificial Sequence
  Description of Artificial Sequence Synthetic polypeptide
  MOD_RES (1306)..(1306): S or T
  MOD_RES (1668)..(1668): D or N
  MOD_RES (1915)..(1915): V or A
MSSDSEMAVFGEAAPYLRKPEKERTEAQNRPFDSKKACFAVDDKEVYVKGMIQNRENDKVTVKTLDDRTITLNTAQVFPMNPPKFDKIEDMAMMTHLHEPAVLYNLKERYAAWMIYTYSGLFCVTVNPYKWLPVYNPEVVMAYRGKKRQEAPPHIFSISDNAYQFMLTDRDNQSILITGESGAGKTVNTKRVIQYFATIAVPGDKKKEPQTGKMQGTLEDQIIQANPLLEAFGNAKTVRNDNSSRFGKFIRIHFGATGKLASADIETYLLEKSRVTFQLSSERSYHIFYQIMSNKKPELIDLLLISTNPFDFPFISQGEVTVASIDDSEELLATDNAIDILGFSSEEKVGIYKLTGAVMHYGNMKFKQKQREEQAEPDGTEVADKAGYLMGLNSAEMLKGLCCPRVKVGNEYVTKGQNVQQVTNSVGALAKAVYEKMFLWMVARINQQLDTKQARQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSANFQKPKPTKGKAEAHFSLVHYAGTVDYNIVGWLDKNKDPLNETVVGLYQKSSLKLLAFLFSNYAGAEAGDCGGSKKGGKKKGSSFQTVSAVFRENLNKLMTNLRSTHPHFVRCLIPNETKTPGVMDHYLVIHQLRCNGVLEGIRICRKGFPSRILYGDFRQRYRILNASAIPEGQFIDSKNASEKLLNSINVDREQYRFGHTKVFFKAGLLGLLEEMRDEKLATLMTRTQAVCRGYLMRVEFKKMMERRDSILCIQDNIRSFMNVKHWPWMNLFFKIQPLLKSAEAEKEMATMKENFKRAQEELARSEARRKELEEKMVSLLQEKNDLQLQVQSETENLMDAEERCEGLIKSKIQLEAKVKELNEKLEEEEEINSELVAKKRNLEDKCFSLKKDIDDLELTLTKVEKEKHATENKVKNLSEEMTALEENISKLTKEKKSLQEAHQQTLDGLQVEEDKVNGLIKINAKLEQQTDDLEGCLEQEKKLRADLGRLKRKLEGDLKISQESITDLENAQQQMEEKLKKKEFEISQLQTKIDGEQVHSLQLQKKIKELQARTEELEEDIEAEHTLRAKLERQRSDLARELEEISERLEEASGATSAQVELNKKREAEFQKMRRDLEEATLQHEATAATLRKKHADSVAELGEQIDNLQRVKQKLQKEKSELKMEMDDMASNIETITKSKSNMERMCRTMEDQFNELKAKDDQQTQLIHDLNMQKARLQTQNGELSHQVEEKESLISQLXKGKQALTQQLEELKRQLEEETKAKNALAHALQSSRHDCDLLREQYEEEQEGKAEVQRALSKANSEVAQWRTKYETDAIQRTEELEEAKKKLAQRLQEAEENTEAVNSKCASLEKTKQRLQGEVDDLMLDLERANTTCATLGKKQRNFDKILADWKQKLDESHAELEAAQKESRSLSTEIFKMKNAYEEVVDQLETLRRENKNLQEEIHDVTEQIAETGKNLQEMEKTKKQVEQEKSDLQVALEEVEGSLEHEESKILRVQLELSQLKSELDRKVAEKDEEMEQLKRNCQRAAEATQSLLEAESRSRKDALRLKKKMEGDLNEMEVHLGHANRQVAETQKHLRTVQGQLKGSQLHLGDALRSXEALKERLAIVGRRNGLLVEELEETKVALEQAERSRRLSEQELQDASDRVQLLLSRCNTSLINIKKKLETNIAQCQAEVENSVQESRNAEEKAKKAITDAAMMAEELKKEQDTSAHLERMKKNLEQTVKDMQHRLDEAEQLALKGDKKQIQKLENRVRELESELDTEQKRGAEALKGAHKYERKVKEMAYQAEEDHKNSLRLQDLVDKLQAKVNAYKRQAEEAEEQANTQLSRCRRVQHELEEAEERXDIAESQVNKLRAKSRDVGSQVRADTWAWLLLKESRDPGAQRSHRESLEAAHWSLLLVMPSQMPPTQQRLL

SEQ ID NO: 15 - protein, 1363 aa, Artificial Sequence
  Description of Artificial Sequence Synthetic polypeptide
  MOD_RES (25)..(25): V or L
  MOD_RES (26)..(26): S or F
MLSFVDTRILLLLAVTSYLATSQHXXZASAGRKGPRGDKGPQGERGPPGPPGRDGEDGPPGPPGPPGPPGLGGNFAAQYDPSKAADFGPGPMGLMGPRGPPGASGPPGPPGFQGVPGEPGEPGQTGPQGPRGPPGPPGKAGEDGHPGKPGRPGERGVAGPQGARGFPGTPGLPGFKGIRGHNGLDGQKGQPGTPGTKGEPGAPGENGTPGQPGARGLPGERGRIGAPGPAGARGSDGSAGPTGPAGPIGAAGPPGFPGAPGAKGEIGPAGNVGPTGPAGPRGEIGLPGSSGPVGPPGNPGANGLPGAKGATGLPGVAGAPGLPGPRGIPGPPGPAGPSGARGLVGEPGPAGAKGESGNKGEPGAAGPPGPPGPSGEEGKRGSNGEPGSAGPPGPAGLRGVPGSRGLPGADGRAGVMGPAGNRGASGPVGAKGPNGDAGRPGEPGLMGPRGLPGQPGSPGPAGKEGPVGFPGADGRVGPIGPAGNRGEPGNIGFPGPKGPTGEPGKPGEKGNVGLAGPRGAPGPEGNNGAQGPPGVTGNQGAKGETGPAGPPGFQGLPGPSGPAGEAGKPGERGLHGEFGVPGPAGPRGERGLPGESGAVGPAGPPGSRGPSGPPGPDGNKGEPGNVGPAGAPGPAGPGGIPGERGVAGVPGGKGEKGAPGLRGDTGATGRDGARGLPGAIGAPGPAGGAGDRGEGGPAGPAGPAGARGIPGERGEPGPVGPSGFAGPPGAAGQPGAKGERGPKGPKGETGPTGAIGPIGASGPPGPVGAAGPAGPRGDAGPPGMTGFPGAAGRVGPPGPAGITGPPGPPGPAGKDGPRGLRGDVGPVGRTGEQGIAGPPGFAGEKGPSGEAGAAGPPGTPGPQGILGAPGILGLPGSRGERGLPGIAGATGEPGPLGVSGPPGARGPSGPVGSPGPNGAPGEAGRDGNPGNDGPPGRDGAPGFKGERGAPGNPGPSGALGAPGPHGQVGPSGKPGNRGDPGPVGPVGPAGAFGPRGLAGPQGPRGEKGEPGDKGHRGLPGLKGHNGLQGLPGLAGQHGDQGPPGNNGPAGPRGPPGPSGPPGKDGRNGLPGPIGPAGVRGSHGSQGPAGPPGPPGPPGPPGPNGGGYEVGFDAEYYRADQPSLRPKDYEVDATLKTLNNQIETLLTPEGSKKNPARTCRDLRLSHPEWSSGFYWIDPNQGCTADAIRAYCDFATGETCIHASLEDIPTKTWYVSKNPKDKKHIWFGETINGGTQFEYNGEGVTTKDMATQLAFMRLLANHASQNITYHCKNSIAYMDEETGNLKKAVILQGSNDVELRAEGNSRFTFSVLVDGCSKKNNKWGKTIIEYRTNKPSRLPILDIAPLDIGGADQEFGLHIGPVCFK

SEQ ID NO: 16 - protein, 1985 aa, Loxodonta africana
MSSDSEMAVFGEAAPYLRKPEKERTEAQNRPFDSKKACFAVDDKEVYVKGMIQNRENDKVTVKTLDDRTITLNTAQVFPMNPPKFDKIEDMAMMTHLHEPAVLYNLKERYAAWMIYTYSGLFCVTVNPYKWLPVYNPEVVMAYRGKKRQEAPPHIFSISDNAYQFMLTDRDNQSILITGESGAGKTVNTKRVIQYFATIAVPGDKKKEPQTGKMQGTLEDQIIQANPLLEAFGNAKTVRNDNSSRFGKFIRIHFGATGKLASADIETYLLEKSRVTFQLSSERSYHIFYQIMSNKKPELIDLLLISTNPFDFPFISQGEVTVASIDDSEELLATDNAIDILGFSSEEKVGIYKLTGAVMHYGNMKFKQKQREEQAEPDGTEVADKAGYLMGLNSAEMLKGLCCPRVKVGNEYVTKGQNVQQVTNSVGALAKAVYEKMFLWMVARINQQLDTKQARQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSANFQKPKPTKGKAEAHFSLVHYAGTVDYNIVGWLDKNKDPLNETVVGLYQKSSLKLLAFLFSNYAGAEAGDCGGSKKGGKKKGSSFQTVSAVFRENLNKLMTNLRSTHPHFVRCLIPNETKTPGVMDHYLVIHQLRCNGVLEGIRICRKGFPSRILYGDFRQRYRILNASAIPEGQFIDSKNASEKLLNSINVDREQYRFGHTKVFFKAGLLGLLEEMRDEKLATLMTRTQAVCRGYLMRVEFKKMMERRDSILCIQDNIRSFMNVKHWPWMNLFFKIQPLLKSAEAEKEMATMKENFKRAQEELARSEARRKELEEKMVSLLQEKNDLQLQVQSETENLMDAEERCEGLIKSKIQLEAKVKELNEKLEEEEEINSELVAKKRNLEDKCFSLKKDIDDLELTLTKVEKEKHATENKVKNLSEEMTALEENISKLTKEKKSLQEAHQQTLDGLQVEEDKVNGLIKINAKLEQQTDDLEGCLEQEKKLRADLGRLKRKLEGDLKISQESITDLENAQQQMEEKLKKKEFEISQLQTKIDGEQVHSLQLQKKIKELQARTEELEEDIEAEHTLRAKLERQRSDLARELEEISERLEEASGATSAQVELNKKREAEFQKMRRDLEEATLQHEATAATLRKKHADSVAELGEQIDNLQRVKQKLQKEKSELKMEMDDMASNIETITKSKSNMERMCRTMEDQFNELKAKDDQQTQLIHDLNMQKARLQTQNGELSHQVEEKESLISQLTKGKQALTQQLEELKRQLEEETKAKNALAHALQSSRHDCDLLREQYEEEQEGKAEVQRALSKANSEVAQWRTKYETDAIQRTEELEEAKKKLAQRLQEAEENTEAVNSKCASLEKTKQRLQGEVDDLMLDLERANTTCATLGKKQRNFDKILADWKQKLDESHAELEAAQKESRSLSTEIFKMKNAYEEVVDQLETLRRENKNLQEEIHDVTEQIAETGKNLQEMEKTKKQVEQEKSDLQVALEEVEGSLEHEESKILRVQLELSQLKSELDRKVAEKDEEMEQLKRNCQRAAEATQSLLEAESRSRKDALRLKKKMEGDLNEMEVHLGHANRQVAETQKHLRTVQGQLKGSQLHLGDALRSNEALKERLAIVGRRNGLLVEELEETKVALEQAERSRRLSEQELQDASDRVQLLLSRCNTSLINIKKKLETNIAQCQAEVENSVQESRNAEEKAKKAITDAAMMAEELKKEQDTSAHLERMKKNLEQTVKDMQHRLDEAEQLALKGDKKQIQKLENRVRELESELDTEQKRGAEALKGAHKYERKVKEMAYQAEEDHKNSLRLQDLVDKLQAKVNAYKRQAEEAEEQANTQLSRCRRVQHELEEAEERADIAESQVNKLRAKSRDVGSQVRADTWAWLLLKESRDPGAQRSHRESLEAAHWSLLLVMPSQMPPTQQRLL

(19) United States
(12) Patent Application Publication
(10) Pub. No.: US 2022/0333081 A1
(43) Pub. Date: Oct. 20, 2022

(54) GENERATION OF CELL-BASED PRODUCTS FOR CONSUMPTION THAT COMPRISE PROTEINS FROM EXOTIC, ENDANGERED, AND EXTINCT SPECIES

(71) Applicant: Upside Foods, Inc., Berkeley, CA (US)

(72) Inventors: Sukhdeep S. DHADWAR, Berkeley, CA (US); Kevin J. KAYSER, Berkeley, CA (US); Nicholas J. GENOVESE, Berkeley, CA (US)

(21) Appl. No.: 17/658,009

(22) Filed: Apr. 5, 2022

Related U.S. Application Data
(60) Provisional application No. 63/171,605, filed on Apr. 7, 2021.

(51) Int. Cl. C12N 5/077; A23L 13/00; A23J 1/02; A23J 3/34; A23J 3/04

(52) U.S. Cl. CPC C12N 5/0659; A23L 13/00; A23J 1/02; A23J 3/341; A23J 3/04; C12N 2533/54

(57) ABSTRACT

The present disclosure relates to methods of preparing cell-based products for consumption that comprise proteins from exotic, endangered, and extinct species, as well as cell-based products for consumption.

### PRIORITY

This application claims priority to U.S. Provisional Application No. 63/171,605, filed Apr. 7, 2021, the entire content of which is incorporated herein by reference.

### SEQUENCE LISTING

The instant application contains a Sequence Listing which has been submitted electronically in ASCII format and is hereby incorporated by reference in its entirety. Said ASCII copy, created on Jun. 10, 2022, is named UF_023_01_WO_SL.txt and is 146,956 bytes in size. Said ASCII copy includes in a single file ASCII copies, created on Apr. 4, 2022, are named (1) ON076011 amino acid sequence (SEQ ID NO:3) (2 KB in size), (2) ON076011 nucleotide sequence (SEQ ID NO:2) (5 KB in size), (3) ON076012 amino acid sequence (SEQ ID NO: 1) (2 KB in size), (4) ON076012 nucleotide sequence (SEQ ID NO: 4) (4 KB in size), (5) ON076013 amino acid sequence (SEQ ID NO: 5) (2 KB in size), and (6) ON076013 nucleotide sequence (SEQ ID NO: 6)(6 KB in size).

### FIELD

This invention is in the field of cell-based products for consumption, in particular, products that comprise proteins derived from exotic, endangered, and/or extinct species. The present disclosure relates to novel consumable products and methods of preparing such consumable products.

### BACKGROUND

As the world's population continues to grow, the need for products for consumption is greater than ever. Given the expanding population, the existing market of conventional products is struggling to meet the demand. In vitro produced cell-based products for consumption have emerged as an attractive option to supplement the demand for conventional products. Moreover, in vitro produced cell-based products for consumption help alleviate several drawbacks linked to conventional products and help address controversial issues associated with, e.g., conventional meat production. Some known factors associated with conventional meat production include low conversion of caloric inputs to edible nutrients, microbial contamination of product, emergence and propagation of veterinary and zoonotic diseases, relative natural resource requirements, and industrial pollutants, such as greenhouse gas emissions and nitrogen waste streams.

Thus, it is an object of the invention to provide methods of preparing in vitro produced cell-based products for consumption. In particular, such cell-based products may comprise peptides and proteins, in addition to lipophilic molecules such as fatty acids, lipids, steroids and their metabolic precursors from exotic, endangered, and extinct species. To date, cell-based products that comprise such molecules have never been prepared, thus demonstrating the novelty of the invention. Cell-based consumption products comprising proteins from exotic, endangered, and/or extinct species may elicit a number of benefits. These may include, for example, generation of unique nutrition profiles and organoleptic profiles never before observed (i.e., from extinct species), to provide options displacing the unsustainable harvest or poaching (i.e., of wild or threatened species), and for generating marketing appeal for new product alternatives that can potentially displace consumption of conventional animal products manufactured by means requiring animal husbandry and slaughter.

### SUMMARY

This invention generally relates to methods of preparing in vitro produced cell-based products for consumption comprising proteins from exotic, endangered, and extinct species. By way of example, the cell-based products may be meat products.

In a first embodiment, DNA constructs encoding for proteins from extinct, exotic, or endangered species may be cloned and expressed in agriculturally or nutritionally-relevant host animal cells. Alternatively, the host cell may come from yeast or bacteria. These transfected cells may then form the basis for generation of cell-based products for consumption. In a preferred embodiment, the DNA constructs derived from the extinct, exotic, or endangered species may be physically integrated into the host animal cell genome via various genomic engineering mechanisms prior to expression. Alternatively, protein from extinct, exotic, or endangered species may be expressed in other hosts, such as eukaryotic, microbial, or yeast systems, and subsequently added into cell-based products for consumption. Such protein may, in separate embodiments, be expressed in vivo.

In a second embodiment, cells from distinct species related by a distant, common ancestor (e.g., an extinct, exotic, or endangered ancestral species) may be fused together to form syncytial heterokaryons. Formation of these syncytial heterokaryons may induce expression of genes from the common ancestral species that were silenced over the course of evolution.

### DESCRIPTION OF DRAWINGS

This patent or application file contains at least one drawing prepared in color. Copies of this patent or patent application publication with color drawing(s) will be provided by the Office upon request and payment of the necessary fee.

FIG. 1 depicts a flow chart exhibiting how food products comprising proteins from extinct, exotic, or endangered species are prepared when DNA constructs encoding genes from such species are transfected into agriculturally or nutritionally-relevant host animal, yeast, or bacterial cells.

FIG. 2 illustrates the trans-dominant modulators involved (avian trans-dominant activators and repressors; crocodilian trans-dominant activators and repressors) upon formation of heterokaryons from crocodilian and avian extant species.

FIG. 3 provides a high-level schematic of how gene expression proceeds upon formation of heterokaryons from crocodilian and avian extant species.

FIGS. 4B-1, 4B-2, 4B-3, and 4B-4 depict Tyrannosaurus Rex (POC2W2.2) and Brachylophosaurus canadensis (P86289.1) collagen peptide sequences mapped by alignment against the protein sequence for chicken collagen 1A1 (COL1A1, NC_052558.1) to form a consensus Dinosaur COL1A1 (ON076011) protein sequence. Shown are COL1A1 amino acid substitutions unique to Tyrannosaurus Rex and Brachylophosaurus canadensis. In particular, substitutions P-406-A and S-407-P from Tyrannosaurus Rex and P-776-S and A-779-P from Brachylophosaurus canadensis have been identified. FIGS. 4B-1-4B-4 disclose SEQ ID NOS 7, 3, and 8-10, respectively, in order of appearance.

FIGS. 4A-1, 4A-2, 4A-3, and 4A-4 depict Tyrannosaurus Rex (P0C2W4.1) and Brachylophosaurus canadensis (P86290.1) collagen peptide sequences mapped by alignment against the peptide sequence for chicken collagen 1A2 (COL1A2, NC_052533.1) to form a consensus Dinosaur COL1A2 (ON076012) protein sequence. Shown are COL1A2 amino acid substitutions unique to Tyrannosaurus Rex and Brachylophosaurus canadensis. In particular, substitutions I-605-P from Tyrannosaurus Rex and A-311-T from Brachylophosaurus canadensis have been identified. FIGS. 4A-1-4A-4 disclose SEQ ID NOS 15, 1, and 11-13, respectively, in order of appearance.

FIGS. 5A-5B depict cloning of dinosaur collagen 1A1 (A) and 1A2 (B) into PhiC31 vectors. Dinosaur-specific COL1A1 (P-406-A, S-407-P, P-776-S and A-779-P) amino acid substitutions were inserted into the chicken COL1A1 reference protein sequence, chicken codon optimized, and designated Dinosaur COL1A1 (ON076011). Dinosaur-specific COL1A2 (I-605-P, A-311-T) amino acid substitutions were inserted into the chicken COL1A2 reference protein sequence, chicken codon optimized, and designated Dinosaur COL1A2 (ON076012). In FIG. 5A, the Dinosaur COL1A1 gene sequence was chicken codon optimized and cloned into a PhiC31 vector under the control of a EF1a promoter. In FIG. 5B, the Dinosaur COL1A2 gene sequence was codon optimized and cloned into a PhiC31 vector under control of a PGK promoter. The Dinosaur COL1A1 and 1A2 vectors were used to develop stable expression in cultured chicken cells for cultured meat.

FIG. 6 depicts cultured chicken cells transfected with Dinosaur collagen 1A1 and 1A2 gene vectors. Dinosaur collagen 1A1 (ON076011) and 1A2 (ON076012) gene vectors or empty vectors were transfected with integrase into cultured chicken cells. A naïve sample served as a control. Cells with stable fluorescent reporter expression were selected by cell sorting and seeded into a 12-well plate and imaged at 10× magnification under bright field, Texas Red (615 nm; RFP) and Green Fluorescence (509 nm; GFP).

FIGS. 7A-7B depict Dinosaur collagen COL1A1 (A) and COL1A2 (B) transcript expression in cultured chicken cells. The relative expression of exogenous Dinosaur COL1A1 (7A) and COL1A2 (7B) was confirmed by quantitative PCR in cultured chicken cells transfected and selected for stable expression of both Dinosaur genes. The relative expression levels of the codon optimized Dinosaur collagen 1A1 and 1A2 genes are based on delta Ct (ΔCt=ΔCt_{Dinosaur COL1A1/2}−ΔCt GAPDH) calculation with endogenous GAPDH mRNA as the reference transcript. The expression of Dinosaur COL1A1 and COL1A2 relative to the expression of endogenous GAPDH is presented as 2^{-ΔCT}. GAPDH has been assigned an expression level of 1. Relative expression levels of COL1A1 (A) and COL1A2 (B) above 1 indicate genes are present at higher levels than GAPDH, while relative expression levels below 1 indicate gene levels are less than GAPDH.

FIG. 8 depicts collagen staining in cultured chicken cells expressing Dinosaur collagen 1A1 and 1A2 genes. Cultured chicken cells were transfected with Dinosaur collagen 1A1 and 1A2 gene vectors accompanied by red fluorescent protein and green fluorescent protein reporters, respectively. Cells were co-transfected with integrase and selected for stable gene expression. 96-well plates were stained with collagen I primary antibody followed by an Alexa Fluor 350 secondary antibody.

FIGS. 9A, 9B, 9C, and 9D depict identification of Mammoth-specific protein coding variations in the myosin heavy chain gene MYH13. Mammoth and elephant genomes were analyzed for protein coding non-synonymous single nucleotide polymorphisms (SNPs) that only exist in Mammoth myosin genes. Elephant genomes examined include African elephant (L_africana_B, L_africana_C, LoxAfr 3.0), Asian elephant (E_maximus_D, E_maximus_E), Forest Elephant (L_cyclotis_A, L_cyclotis_F), Straight tusked elephant (P_antiquus_N, P_antiquus_O). Mammoth genomes included in the analysis were Woolly Mammoth (Wrangle, Oimyakon, M_primigenius_G, M_primigenius_H, M_primigenius_S, M_primigenius_V) and Columbian Mammoth (M_columbi_U). Mammoth-specific protein coding non-synonymous SNPs were identified in myosin heavy chain gene MYH13 to include T-1306-S, N-1668-D, A-1914-V. These amino acid substitutions did not exist in any elephant genomes analyzed. The identified amino acid substitutions were applied to the Loxodonta africana MYH13 sequence (XP_003416837.1) to create Mammoth MYH13 (ON076013). FIGS. 9A-9D disclose SEQ ID NOS 14, 5 and 16, respectively, in order of appearance.

FIGS. 10A-10B depict a codon optimized Mammoth MYH13 gene. The mammoth myosin heavy chain gene (MYH13) containing amino acids 1914-V, 1668-D, and 1306-S was created by mapping the amino acid substitutions to the Loxodonta africana reference genome. The nucleotide sequence was chicken codon optimized for expression in cultured chicken cells. FIGS. 10A-10B disclose SEQ ID NO:6.

FIG. 11 depicts a mammoth MYH13 gene inserted into a PhiC31 vector. The mammoth-specific MYH13 containing 1914-V, 1668-D, and 1306-S amino acids was encoded in the Loxodonta africana MYH13 reference sequence. The nucleotide sequence was chicken codon optimized and cloned into a PhiC31-RFP vector for stable expression in cultured chicken cells developed for cultured meat.

FIG. 12 depicts cultured chicken cells transfected with a Mammoth MYH13 gene vector (ON076013). Mammoth MYH13 was expressed along with a red fluorescent reporter (RFP) in cultured chicken cells using the PhiC31 vector system. Mammoth MYH13 gene vector or empty vector was transfected with integrase into cultured chicken cells. A naïve sample served as a control. Cells with stable expression were selected and seeded into a 12-well plate and imaged at 10× magnification under bright field, Texas Red (615 nm; RFP) and Green Fluorescence (509 nm; GFP). The naïve sample (non-transfected control; top panel) showed no fluorescence when imaged in the RFP channel. An RFP empty vector (PhiC31-RFP) is depicted in the middle panel.

FIG. 13 depicts Mammoth myosin MYH13 transcript expression in cultured chicken cells. The relative expression of exogenous Mammoth MYH13 was confirmed by quantitative PCR in cultured chicken cells transfected and selected for stable expression. The relative expression level of codon optimized Mammoth MYH13 mRNA is based on delta Ct (ΔCt=ΔCt_{Mammoth MYH13}−ΔCt GAPDH) calculation with endogenous GAPDH mRNA as the reference transcript. The expression of Mammoth MYH13 relative to the expression of endogenous GAPDH is presented as 2^{-ΔCT}.

FIG. 14 depicts Mammoth MYH13 staining in cultured chicken cells expressing the Mammoth MYH13 gene. Cultured chicken cells were transfected with Mammoth MYH13 gene vector accompanied by a red fluorescent protein reporter. Cells were co-transfected with integrase and selected for stable gene expression. 96-well plates were stained with MYH13 primary antibody followed by an Alexa Fluor 488 secondary antibody. Cell nuclei were stained with DAPI. GAPDH has been assigned an expression level of 1. Relative expression levels of MYH13 above 1 indicate genes are present at higher levels than GAPDH, while relative expression levels below 1 indicate gene levels are less than GAPDH.

### DETAILED DESCRIPTION OF THE INVENTION

Provided herein are methods and compositions related to the in vitro production of cell-based products for consumption comprising proteins from exotic, endangered, and extinct species. For further detail regarding in vitro production of cell-based products for consumption, please reference U.S. Publication No. 2021/0235733 A1, the entire content of which is incorporated herein.

Before describing particular embodiments in detail, it is to be understood that the disclosure is not limited to the particular embodiments described herein, which can vary. It is also to be understood that the terminology used herein is for the purpose of describing particular illustrative embodiments only and is not intended to be limiting unless otherwise defined. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the invention and how to make and use them. The scope and meaning of any use of a term will be apparent from the specific context in which the term is used. As such, the definitions set forth herein are intended to provide illustrative guidance in ascertaining particular embodiments of the invention, without limitation to particular compositions or biological systems.

As used in the present disclosure and the appended claims, the singular forms “a,” “an” and “the” include plural references unless the content clearly dictates otherwise.

Throughout the present disclosure and the appended claims, unless the context requires otherwise, the word “comprise”, or variations such as “comprises” or “comprising”, will be understood to imply the inclusion of a stated element or group of elements but not the exclusion of any other element or group of elements.

Unless specific definitions are provided, the nomenclature utilized in connection with, and the laboratory procedures and techniques of, molecular biology, cell biology, analytical chemistry, and synthetic organic chemistry described herein are those well-known and commonly used in the art. Standard techniques may be used for recombinant technology, molecular, biological, microbiological, chemical syntheses, and chemical analyses.

### Generation of Cell-Based Products for Consumption

Provided herein are methods to produce in vitro cell-based products for consumption.

### Cells

The cell-based products for consumption of the disclosure are compositions produced by the in vitro culturing of naturally occurring, transgenic, or modified animal cells in culture. In alternative embodiments, cell-based products for consumption may be prepared by culture in various recombinant protein expression systems, such as microbial or yeast systems.

The cells used in the methods of the present disclosure can be primary cells, or modified cell lines, such as adapted or immortalized cell lines. The methods provided herein are applicable to any metazoan cell in culture. Generally, the cells are from any metazoan species whose tissues are suitable for dietary consumption and demonstrate the capacity for skeletal muscle tissue specification.

In some embodiments, the cells are derived from any non-human animal species intended for human or non-human dietary consumption, for example, cells of avian, ovine, caprine, porcine, bovine, and piscine origin, or, in other words, cells of livestock, poultry, avian, game, or aquatic species.

In some embodiments, the cells are from livestock, such as domestic cattle, pigs, sheep, goats, camels, water buffalo, rabbits and the like. In some embodiments, the cells are from poultry, such as domestic chicken, turkeys, ducks, geese, pigeons and the like. In some embodiments, the cells are from game species, such as wild deer, gallinaceous fowl, waterfowl, hare and the like. In some embodiments, the cells are from aquatic species or semi-aquatic species harvested commercially from wild fisheries or aquaculture operations, or for sport, including certain fish, crustaceans, mollusks, cephalopods, cetaceans, crocodilians, turtles, frogs and the like.

In some embodiments, the cells are from exotic, conserved or endangered animal species. In some embodiments, the cells are from Gallus gallus, Gallus domesticus, Bos taurus, Sous scrofa, Meleagris gallopavo, Anas platyrynchos, Salmo salar, Thunnus thynnus, Ovis aries, Coturnix, Capra aegagrus hircus, or Homarus americanus.

In some embodiments, the cells are primary stem cells, self-renewing stem cells, embryonic stem cells, pluripotent stem cells, induced pluripotent stem cells, or trans-differentiated pluripotent stem cells.

In some embodiments, the cells are modifiable by a genetic switch to induce rapid and efficient conversion of the cells to skeletal muscle for cultured production.

In some embodiments, the cells are myogenic cells destined to become muscle or muscle-like cells. In some embodiments, the myogenic cells are natively myogenic, e.g., myoblasts. Natively myogenic cells include, but are not limited to, myoblasts, myocytes, satellite cells, side population cells, muscle derived stem cells, mesenchymal stem cells, myogenic pericytes, or mesangioblasts.

In some embodiments, cells are of skeletal muscle lineage. Cells of skeletal muscle lineage include myoblasts, myocytes, and skeletal muscle progenitor cells, also called myogenic progenitors that include satellite cells, side population cells, muscle derived stem cells, mesenchymal stem cells, myogenic pericytes, and mesangioblasts.

In some embodiments, the cells are non-myogenic, and such non-myogenic cells can be programmed to be myogenic, for example the cells may comprise fibroblasts modified to express one or more myogenic transcription factors. In exemplary embodiments, the myogenic transcription factors include MYOD1, MYOG, MYF5, MYF6, PAX3, PAX7, paralogs, orthologs, and genetic variants thereof. In some embodiments, the cells are modified to express one or more myogenic transcription factors as described in a PCT Publication No. WO/2015/066377, incorporated by reference herein in its entirety.

In some embodiments, the cells comprise a mixture of cell populations described herein, e.g., a mixture of fibrogenic cells and myogenic cells in co-culture, e.g., a mixture of fibroblasts and myoblasts in co-culture. In some embodiments, the cells used for the in vitro production of cell-based products for consumption are a mixture of fibroblasts and myoblasts in a suspension co-culture. In some embodiments the cells used for the in vitro production of cell-based products for consumption are a mixture of fibroblasts and myoblasts in an adherent co-culture. In some embodiments, the co-culture can further comprise adipocytes.

In some embodiments, the cells are in either a suspension culture or adherent co-culture, and comprise a mixture of fibroblasts and myoblasts, wherein the ratio of the fibroblasts to myoblasts (designated as F and M) ranges from about 5F:95M to about 95F:5M. In exemplary embodiments, the ratio of the fibroblasts to myoblasts is about 5F:95M, 10F:90M, 15F:85M, 20F:80M, 25F:75M, 30F:70M, 35F:65M, 40F:60M, 45F:55M, 50F:50M, 55F:45M, 60F:40M, 65F:35M, 70F:30M, 75F:25M, 80F:20M, 85F:15M, 90F:10M, or even about 95F:5M.

In some embodiments, the cells are genetically modified to inhibit a pathway, e.g., the HIPPO signaling pathway. Exemplary methods to inhibit the HIPPO signaling pathway as described in PCT Publication No. WO/2018/208628, incorporated by reference herein in its entirety.

In some embodiments, the cells are modified to express telomerase reverse transcriptase (TERT) and/or inhibit cyclin-dependent kinase inhibitors (CKI). In some embodiments, the cells are modified to express TERT and/or inhibit cyclin-dependent kinase inhibitors as described in PCT Publication No. WO 2017/124100, incorporated by reference herein in its entirety.

In some embodiments, the cells are modified to express glutamine synthetase (GS), insulin-like growth factor (IGF), and/or albumin. Exemplary methods of modifying cells to express GS, IGF, and/or albumin are described in PCT Publication No. WO/2019/014652, which is incorporated by reference herein in its entirety.

In some embodiments, the cells may comprise any combination of the modifications described herein.

### Cultivation Infrastructure

As referred to herein, a cultivation infrastructure refers to the environment in which the cells are cultured or cultivated to provide a two-dimensional or three-dimensional product for consumption.

A cultivation infrastructure may be a roller bottle, a tube, a cylinder, a flask, a petri-dish, a multi-well plate, a dish, a vat, an incubator, a bioreactor, an industrial fermenter and the like.

While the cultivation infrastructure itself may have a three-dimensional structure or shape, the cells cultured in the cultivation infrastructure may form a monolayer of cells. Compositions and methods of the present disclosure can promote a three-dimensional growth of metazoan cells in the cultivation infrastructure to provide a scaffold-less self-assembly of a three-dimensional cellular biomass.

A three-dimensional cultivation infrastructure may be sculpted into different sizes, shapes, and forms, as desired, to provide the shape and form for the muscle cells to grow and resemble different types of muscle tissues such as steak, tenderloin, shank, chicken breast, drumstick, lamb chops, fish fillet, lobster tail, etc. The three-dimensional cultivation infrastructure may be made from natural or synthetic biomaterials that are non-toxic so that they may not be harmful if ingested. Natural biomaterials may include, for example, collagen, fibronectin, laminin, or other extracellular matrices. Synthetic biomaterials may include, for example, hydroxyapatite, alginate, polyglycolic acid, polylactic acid, or their copolymers. The three-dimensional cultivation infrastructure may be formed as a solid or semisolid support.

A cultivation infrastructure can be of any scale and support any volume of cellular biomass and culturing reagents. In some embodiments, the cultivation infrastructure ranges from about 10 μL to about 100,000 L. In exemplary embodiments, the cultivation infrastructure is about 10 μL, about 100 μL, about 1 mL, about 10 mL, about 100 mL, about 1 L, about 10 L, about 100 L, about 1000 L, about 10,000 L, or even about 100,000 L.

In some embodiments, the cultivation infrastructure comprises a substrate. A cultivation infrastructure may comprise a permeable substrate (e.g., permeable to physiological solutions) or an impermeable substrate (e.g., impermeable to physiological solutions). The substrate can be flat, concave, or convex. The substrate may be textured so as to promote cell growth and cell sheet attachment.

In some embodiments, the culturing of cells in the cultivation infrastructure can induce the production of extracellular matrix (ECM) that may act as an autologous scaffold to direct three-dimensional cellular growth, e.g., to direct attachment, proliferation and hypertrophy of cells on a plane perpendicular to the substrate.

In some embodiments, the cultivation infrastructure may not comprise an exogenously added scaffold to promote self-assembly of a three-dimensional cellular biomass. In some embodiments, the cultivation infrastructure may not comprise exogenous scaffolds such as a hydrogel or soft agar.

### Culturing Conditions

The culturing conditions for the generation of cell-based products for consumption are generally aseptic, and sterile.

Cells can be grown in an adherent culture format to form a cell sheet or can be grown in a suspension culture format to form a cell pellet. Table 1, shown below, provides exemplary culture methods for the various products that can be produced in vitro.

TABLE 1
Cell culture methods used to generate in vitro produced cell-based meat

| | | Culture | | |
|---|---|---|---|---|
| | | Condition | | |
| | | Cell Type(s) | Culture | |
| Method # | Sample ID | (ratio) | format | Base media |
| 1 | A. platyrhynchos (duck) | Co-culture | Adherent | DMEM-F12 with |
| | fibroblast/myoblast tissue 1 | F/M (50/50) | | FBS (High), BS (High), |
| | | | | CS (Low), HS (Low) |
| 2 | A. Platyrhynchos (duck) | Monoculture F | Adherent | DMEM-F12 with |
| | fibroblast tissue 1 | | | FBS (High), BS (High), |
| | | | | CS (Low), HS (Low) |
| 3 | Bos (Cow) | Monoculture F | Adherent | DMEM-F12 with |
| | fibroblast tissue 1 | | | FBS (High), BS (High), |
| | | | | CS (Low), HS (Low) |
| 4 | Gallus (chicken) | Monoculture F | Adherent | DMEM-F12 with |
| | fibroblast tissue 1 | | | FBS (High), CS (Low) |
| 5 | Gallus (chicken) | Monoculture F | Adherent | DMEM-F12 with |
| | fibroblast tissue 2 | | | CS (High), BS (Low) |
| 6 | Gallus (chicken) | Monoculture F | Adherent | DMEM-F12 with |
| | fibroblast tissue 3 | | | CS (High), BS (High) |
| 7 | Gallus (chicken) | Monoculture F | Adherent | DMEM-F12 with |
| | fibroblast tissue 4 | | | BS (High), CS (Low) |
| 8 | Gallus (chicken) | Monoculture F | Adherent | DMEM-F12 with 10% FBS |
| | fibroblast cells 1 | | | |
| 9 | Gallus (chicken) | Co-culture | Adherent | DMEM-F12 with |
| | fibroblast/myoblast tissue 1 | F/M (30/70) | | FBS (High), CS (Low) |
| 10 | Gallus (chicken) | Monoculture F | Adherent | DMEM-F12 with |
| | fibroblast tissue 5 | | | BS (High), CS (Low) |
| 11 | Gallus (chicken) | Monoculture M | Suspension | DMEM-F12 with |
| | myoblast cells 1 | | | BS (High), CS (Low) |
| 12 | Gallus (chicken) | Co-culture | Adherent | DMEM-F12 with |
| | fibroblast/myoblast tissue 2 | F/M (30/70) | | BS (High), CS (Low |
| 13 | Gallus (chicken) | Co-culture | Adherent | DMEM-F12 with |
| | fibroblast/myoblast tissue 3 | F/M (50/50) | | BS (High), CS (Low) |
| 14 | Gallus (chicken) | Co-culture | Adherent | DMEM-F12 with |
| | fibroblast/myoblast tissue 4 | F/Monoclonal | | BS (High), CS (Low |
| | | M (50/50) | | |
| 15 | Gallus (chicken) | Co-culture | Adherent | Chemically-defined media |
| | fibroblast/myoblast tissue 5 | F/Monoclonal | | with BS (low) |
| | | M (70/30) | | |
| 16 | Gallus (chicken) | Monoculture M | Suspension | Chemically defined media |
| | myoblast cells 2 | | | formula. No serum |
| 17 | Gallus (chicken) | Monoculture M | Suspension | SMEM-F12 with |
| | myoblast cells 3 | | | BS (high), CS (low) |

In some embodiments, the media is substantially free of serum or other components derived from an animal.

Accordingly, an exemplary method of producing in vitro produced cell-based meat comprises: (a) providing fibroblasts and/or myoblasts from a non-human organism; (b) culturing the fibroblasts and/or myoblasts in media under conditions under which the fibroblasts and/or myoblasts grow in either suspension culture or adherent culture, wherein the media is substantially free of serum and other components derived from an animal.

In some embodiments, the cells are grown in a suspension culture, e.g., in a shake flask, and the product of the culture is centrifuged, yielding a cell pellet. In other embodiments, the cells are grown in adherent culture, and the product of the culture is a cell sheet.

### Formulation

The cell-based products for consumption of the disclosure may be processed into any variety of products including, but not limited to, cell-based processed food products, cell-based meat products, cell-based beverage products, additives, ingredients, nutritional supplements, vitamins, nutraceuticals, broths, flavorings, fractionations, concentrates, decoctions, isolates, extracts, tinctures, dyes, oils, soaps, detergents, lubricants, fragrances, cosmetics, medications, emollients, topical ointments, or any other suitable edible or consumable product. Exemplary cell-based products of the disclosure include cell-based meat products, such as, for example, avian meat products, chicken meat products, duck meat products, and bovine meat products.

### Characteristics of Cell-Based Products for Consumption

Provided herein are in vitro produced cell-based products for consumption comprising a number of unique features that allow them to be distinguished from conventional products, for example, conventional meat products, which can involve the slaughter or demise of live animals. The in vitro methods can also be tailored to achieve desired traits such as health and sensory benefits.

### Hormones

As compared to conventional products, the in vitro produced cell-based products of the disclosure can comprise a significantly lower amount of steroid hormones. For example, using the in vitro culturing methods described, there need not be any exogenous hormones added into the culture thus resulting in lower or non-existent hormonal levels in a resulting cell-based meat product. Accordingly, in some embodiments, the cell-based product is substantially free of steroid hormones (i.e., contains little or no steroid hormones). This is in contrast to products from animals raised for conventional meat production, which are often fed or otherwise administered exogenous hormones.

Accordingly, in some embodiments, the cell-based product of the disclosure comprises no more than about 1 ug, 0.5 ug, 0.1 ug, 0.05 ug, 0.01 ug, 0.005 ug, or even about 0.001 ug steroid hormone/kg dry mass of cell-based product. In some embodiments, the cell-based product comprises no more than about 1 ug, 0.5 ug, 0.1 ug, 0.05 ug, 0.01 ug, 0.005 ug, or even about 0.001 ug progesterone/kg dry mass of cell-based product. In some embodiments, the cell-based product comprises no more than about 1 ug, 0.5 ug, 0.1 ug, 0.05 ug, 0.01 ug, 0.005 ug, or even about 0.001 ug testosterone/kg dry mass of cell-based product. In some embodiments, the cell-based product comprises no more than about 0.05 ug, 0.01 ug, 0.005 ug, or even about 0.001 ug estradiol/kg dry mass of cell-based product. In exemplary embodiments, the cell-based product comprises no more than about 35 ng estradiol/kg dry mass of cell-based product.

### Microbial Contamination

Using the sterile, laboratory-based in vitro culturing methods described, the cell-based product is substantially free of microbial contaminants. “Substantially free” means that the concentration of microbes or parasites is below a clinically significant level of contamination, i.e., below a level wherein ingestion could lead to disease or adverse health conditions. Such low levels of contamination allow for an increased shelf life. This is in contrast to products from animals raised for conventional meat production. As used herein, microbial contamination includes, but is not limited to, bacteria, fungi, viruses, prions, protozoa, and combinations thereof. Harmful microbes may include coliforms (fecal bacteria), E. coli, yeast, mold, Campylobacter, Salmonella, Listeria, and Staph.

In addition, cells grown in culture may be substantially free from parasites such as tapeworms that infect cells of live animals and that are transferred to humans through consumption of insufficiently cooked meat.

### Antibiotics

Relative to conventional products, in vitro produced cell-based products of the disclosure comprise a significantly lower amount of antibiotics, or are substantially free of antibiotics, or are free of antibiotics entirely. For example, using the in vitro culturing methods described herein, the use of antibiotics in culture can be controlled or eliminated, thus resulting in lower or non-existent antibiotic levels in the resulting cell-based product. Accordingly, in some embodiments, the cell-based product is substantially free of antibiotics (i.e., contains little or no antibiotics). This is in contrast to animals raised for conventional meat production, which are often fed or otherwise administered exogenous antibiotics.

Accordingly, in some embodiments, the cell-based product of the disclosure comprises no more than about 100 ug antibiotics/kg dry mass of cell-based product, 90 ug antibiotics/kg dry mass of cell-based product, 80 ug antibiotics/kg dry mass of cell-based product, 70 ug antibiotics/kg dry mass of cell-based product, 60 ug antibiotics/kg dry mass of cell-based product, 50 ug antibiotics/kg dry mass of cell-based product, 40 ug antibiotics/kg dry mass of cell-based product, 30 ug antibiotics/kg dry mass of cell-based product, 20 ug antibiotics/kg dry mass of cell-based product, 10 ug antibiotics/kg dry mass of cell-based product, 5 ug antibiotics/kg dry mass of cell-based product, 1 ug antibiotics/kg dry mass of cell-based product, 0.5 ug antibiotics/kg dry mass of cell-based product, 0.1 ug antibiotics/kg dry mass of cell-based product, 0.05 ug antibiotics/kg dry mass of cell-based product, or even about 0.01 ug/kg of antibiotics/kg dry mass of cell-based product.

### Lipids

As compared to conventional products, the in vitro produced cell-based products of the disclosure comprise a lower average total lipid (fat) content. For example, cell-based meat generally has an average total fat content between about 0.5% to about 5.0%, whereas the fatty acid content in conventional meat varies widely and can range from about 3% to about 18%, depending on the cut of meat.

Accordingly, in some embodiments, the cell-based product of the disclosure comprises an average total fat content of about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0%, when measured as a % of total wet mass of the cell-based product. A lower fat content provides a lower caloric content, as well as other related health benefits, when compared to conventional products.

The methods provided herein can alter specific fatty acid profiles to achieve desired flavor characteristics or fatty acid profiles of the product. The lower levels of fatty acids in the cell-based product of the disclosure also promotes an increased shelf life, for example by leading to lower levels of fatty oxidation in the product.

### Amino Acids

The cell-based meat product of the disclosure generally comprises about 50 g to about 95 g by weight of amino acids per 100 g dry mass.

### Vitamin E Content

As compared to conventional products, the in vitro produced cell-based products of the disclosure comprise a higher Vitamin E (αTocopherol) content. In some embodiments, the cell-based product of the disclosure comprises at least about 0.5 mg, at least about 0.6 mg, at least about 0.7 mg, at least about 0.8 mg, at least about 0.9 mg, or at least about 1.0 mg/Vitamin E/100 g wet mass of cell-based product.

### Moisture Content

The cell-based product of the disclosure generally has a moisture content of about 65% to about 95%.

### Architecture of Cell-Based Meat

By way of example, cell-based meat, unless otherwise manipulated to include, does not include vascular tissues, such as veins and arteries, whereas conventional meat does contain such vasculature, and contains the blood found in the vasculature. Accordingly, in some embodiments, the cell-based meat does not comprise any vasculature.

Likewise, cell-based meat, although composed of muscle or muscle-like tissues, unless otherwise manipulated to include, does not comprise functioning muscle tissue. Accordingly, in some embodiments, the cell-based meat does not comprise functioning muscle tissue.

It is noted that features such as vasculature and functional muscle tissue can be further engineered into the cell-based meat produced from this disclosure, should there be a desire to do so.

### Supplementation

In other embodiments, other nutrients, such as vitamins, may be added to increase the nutritional value of the cell-based product. For example, this may be achieved through the exogenous addition of the nutrients to the growth medium or through genetic engineering techniques.

### Shelf Life

A significant portion of meat and meat products are spoiled every year. It is estimated that approximately 3.5 billion kg of poultry and meat are wasted at the consumer, retailer and foodservice levels which have a substantial economic and environmental impact (Kantor et al. (1997)). A significant portion of this loss is due to microbial spoilage.

Conventional meat is perishable and has a relatively short shelf life stability (interchangeably referred to as simply “shelf life” herein). The shelf life is the amount of time a food remains fit for human consumption. The composition of conventional meat and the conditions used to slaughter and harvest the meat create favorable growth conditions for various microorganisms including fecal bacteria (e.g., coliform bacteria). Meat is also very susceptible to spoilage due to chemical, oxidative and enzymatic activities. It is generally regarded that microbial growth, oxidation and enzymatic autolysis are three mechanisms responsible for the spoilage of meat. The breakdown of fat, protein and carbohydrates of meat results in the development of off-odors and off-flavor and these the off-odors and off-flavors make the meat objectionable for human consumption. Depending on the species and method of harvest, conventional meat products are not safe to consume after a relatively short period of storage time. For example, chicken should be cooked within a few days of processing and/or purchasing. Cooked poultry can be safely stored in the fridge for only 4 days and the freezer for up to 4 months. It is, therefore, necessary to control meat spoilage in order to increase its shelf life and maintain its nutritional value, texture and flavor.

In vitro produced cell-based meat, through its method of production and composition, produces a meat product that has extended shelf life compared to conventional meat products and does not require the addition of preservative agents to obtain the shelf-life stability. The composition of cell-based meat is such that fewer off-odors and off-flavors are detected. In addition, the manufacturing methods used to produce in vitro cell-based meat require clean and aseptic conditions. These conditions ensure that microbial cell counts in both harvested products and subsequent food processing are low. These multiple factors contribute to extended shelf-life stability of in vitro cell-based meat.

The shelf life due to spoilage of the cell-based meat of the disclosure is enhanced relative to conventional meat. This is the case both at room temperature (about 25° C.) and at colder temperatures (e.g. about 4° C.). The increased shelf life is associated with reduced contamination, composition of the cell-based meat, reduced degradation of the cell-based meat and slower rates of change in color, spoilage, smell and flavor of the cell-based meat.

Without being bound to theory or mechanism, there is a decrease in total fatty acid content in the cell-based meat prepared via the methods described herein, as compared to conventional meat, resulting in lower levels of fatty acid oxidation, leading to slower rates of change in the color, smell, or flavor of the meat.

Without being bound to theory or mechanism, there is a decrease in the number of lipases in the cell-based meat prepared via the methods described herein, as compared to conventional meat, resulting in lower levels of fatty acid breakdown, leading to slower rates of change in the color, smell, or flavor of the meat.

Without being bound to theory or mechanism, due to the absence of vasculature in the cell-based meat prepared via the methods described herein, when compared to conventional meat, there is less oxygen present, resulting in lower levels of fatty acid oxidation and the growth of aerobic bacteria, leading to reduced microbial contamination levels, and leading to slower rates of change in the color, smell, or flavor of the meat.

Without being bound to theory or mechanism, due to the absence of functional muscle tissue (e.g., myoglobin) in the cell-based meat prepared via the methods described herein when compared to conventional meat, there is less oxygen present, resulting in lower levels of fatty acid oxidation and the growth of aerobic bacteria, leading to reduced microbial contamination levels, and leading to slower rates of change in the color, smell, or flavor of the meat.

Without being bound to theory or mechanism, due to higher amounts of Vitamin E in the cell-based meat prepared via the methods described herein when compared to conventional meat, there are higher levels of antioxidant activity, resulting in protection against fatty acid oxidation, and leading to slower rates of change in the color, smell, or flavor of the meat.

Accordingly, in some embodiments, as compared to conventional meat, the shelf life of cell-based meat is increased by at least about 1.5×, at least about 2×, at least about 2.5×, at least about 3×, at least about 3.5×, at least about 4×, at least about 4.5×, at least about 5×, at least about 5.5×, at least about 6×, at least about 6.5×, at least about 7×, at least about 7.5×, at least about 8×, at least about 8.5×, at least about 9×, at least about 9.5×, or even at least about 10×. The shelf-life increases are observed both at about 4° C., and about 25° C., and all temperatures in between inclusive of the endpoints.

Cell-Based Products for Consumption Comprising Proteins from Exotic, Endangered, or Extinct Species

In preferred embodiments of the invention, the cell-based products for consumption may comprise proteins from exotic, endangered, or extinct species. In a particular embodiment, the cell-based products for consumption may be cell-based meat products. The proteins from exotic, endangered, or extinct species may comprise, but are not limited to, myosin, actin, creatine kinase, tropomyosin, fibronectin, collagen, myoglobin, or keratin. The proteins may come from such species as mammoth, mastodon, saber-tooth tiger, hippopotamus, lion, dinosaur, rhinoceros, elephant, passenger pigeon, or shark.

In a preferred first embodiment, DNA, RNA, or protein sequences from any of the above species may be identified by single-nucleotide polymorphisms (SNPs) and compared to a reference genome for an agriculturally- or nutritionally-relevant host animal, bacteria, or yeast. Identified regions for DNA sequences may be coding or non-coding. By way of example, a protein sequence for dinosaur collagen may be identified and mapped to a reference chicken genome. Or, alternatively, nuclear or mitochondrial genomic sequences for woolly mammoths or mastodons may be identified and mapped to a reference elephant genome.

In an alternative embodiment, protein may be expressed in microbial or yeast systems and subsequently purified, before being added into cell-based products for consumption during formulation. In a separate embodiment, protein may be expressed in vivo, for instance, in an embryo, such as a fertilized chicken egg.

In certain embodiments, the agriculturally- or nutritionally-relevant host animal may have common ancestry with the exotic, extinct, or endangered species and may be familiar to human consumption. In preferred embodiments, the entity for consumption which comes from host animals may be beef, pork, chicken, seafood, fish, venison, goose, turkey, duck. Such hosts may be identified as “agriculturally-relevant hosts”. With respect to common ancestry between host animals and exotic, extinct, or endangered species, by way of example, it is known that dinosaurs are a close common ancestor to poultry. In one instance, dinosaur collagen may be cloned into a chicken, squab, or turkey host line. Alternatively, mammals may be selected for expression of mammalian-derived proteins. In one instance, for example, proteins from an endangered red stag species may be expressed in an elk line.

RNA or protein from any of the above species may then be back-translated into DNA sequences, which are then cloned in plasmids or vectors that are functional in host species. In certain embodiments, the plasmids or vectors may include a promoter DNA sequence (e.g., a cis-genic promoter) upstream of a cloning site designed to receive the back-translated DNA sequences. The plasmids or vectors may then be transfected by standard transfection methods into selected agriculturally- or nutritionally-relevant host animal cells for culture, such as, for example, host chicken, bacteria, or yeast cells, in order to enable incorporation of DNA sequences encoding for proteins from exotic, endangered, or extinct species into such host animal cells. In some embodiments, delivery of expression vectors into host cells may include any suitable mechanism, including, but not limited to, CRISPR, Zinc Fingers, Talens, PhiC31, Piggybac, artificial chromosomes, or viral vectors. In a preferred further embodiment, the proteins may be muscle-derived. By way of example, an amino acid sequence of a skeletal muscle myosin protein from mammoth may be determined and the DNA for that protein transfected into a host cell.

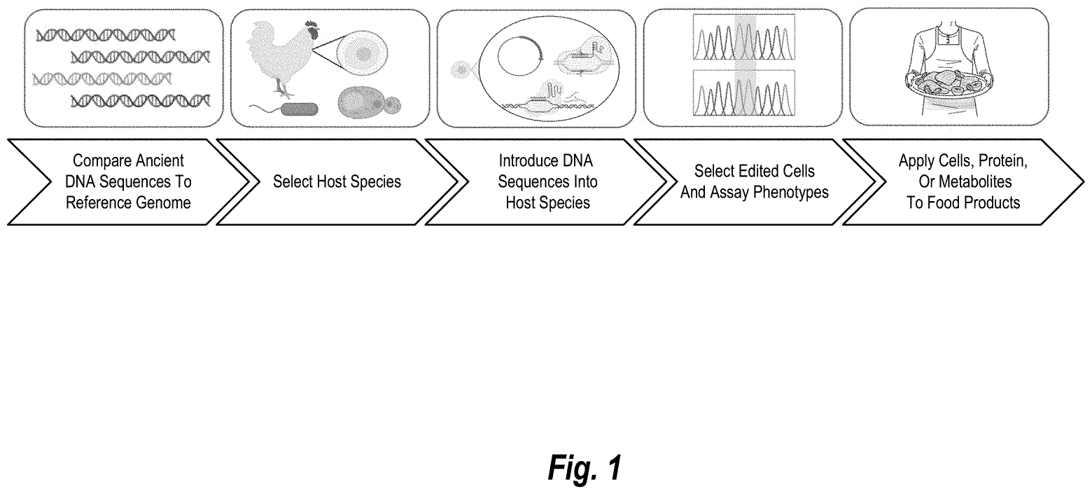

Genetic modifications, such as insertions or deletions, may then be introduced into the host genome to create identified unique SNPs or specific protein products. Genome modification may comprise any suitable mechanism including, but not limited to, CRISPR, Zinc Fingers, Talens, PhiC31, Piggybac, artificial chromosomes, or viral vectors. Alternatively, expression or repression of the host genome may be achieved by epigenetic activation or repression by any suitable mechanism, such as, for example, CRISPR, Zinc Fingers, or relevant transcription factors. Integrated DNA sequences from extinct, exotic, or endangered species may then be expressed (e.g., mRNA-transcribed and translated into protein) by the host animal cell machinery. In a preferred further embodiment, the DNA sequences may be codon-optimized to allow for efficient protein expression in host cells. Transfected host cells may then be selected and evaluated phenotypically for cell growth, gene expression, protein production, or production. Host animal cells demonstrating successful transfection and DNA expression may then be isolated and cloned to produce cell-based products for consumption, as described above. A flow chart depicting the above process can be seen in FIG. 1.

In a further embodiment, the host native protein that corresponds to that expressed from exotic, endangered, or extinct species may be knocked out, deleted, or repressed via any known genetic recombination mechanism, such as CRISPR or homing endonucleases. Alternatively, the host native protein may be left unchanged.

In a further embodiment, the transfected animal cell may comprise a mix of host native protein and transfected exotic/endangered/extinct protein. The mix, for instance, may range from 95% host protein:5% transfected protein, 85% host protein:15% transfected protein, 60% host protein:40% transfected protein, 50% host protein:50% transfected protein, 40% host protein:60% transfected protein, 15% host protein:85% transfected protein, or 5% host protein:95% transfected protein, or any other suitable range not listed above.

In a further embodiment, the host protein may be completely knocked out such that the host animal cell comprises 100% transfected protein. For example, in a particular embodiment, collagen from a dinosaur species may be cloned and expressed in a cultured chicken cell and the native chicken collagen knocked out such that the cultured chicken cell only produces dinosaur collagen.

Food products may then be created from resulting, successfully transfected host cells. Alternatively, food products may be created from expressed proteins or metabolites from exotic, extinct, or endangered species. By way of example, dinosaur collagen may be expressed in cultured chicken host cells to produce cell-based meat or, alternatively, purified from yeast fermentation and added to food products. In alternative embodiments, the dinosaur collagen may be employed to produce jello.

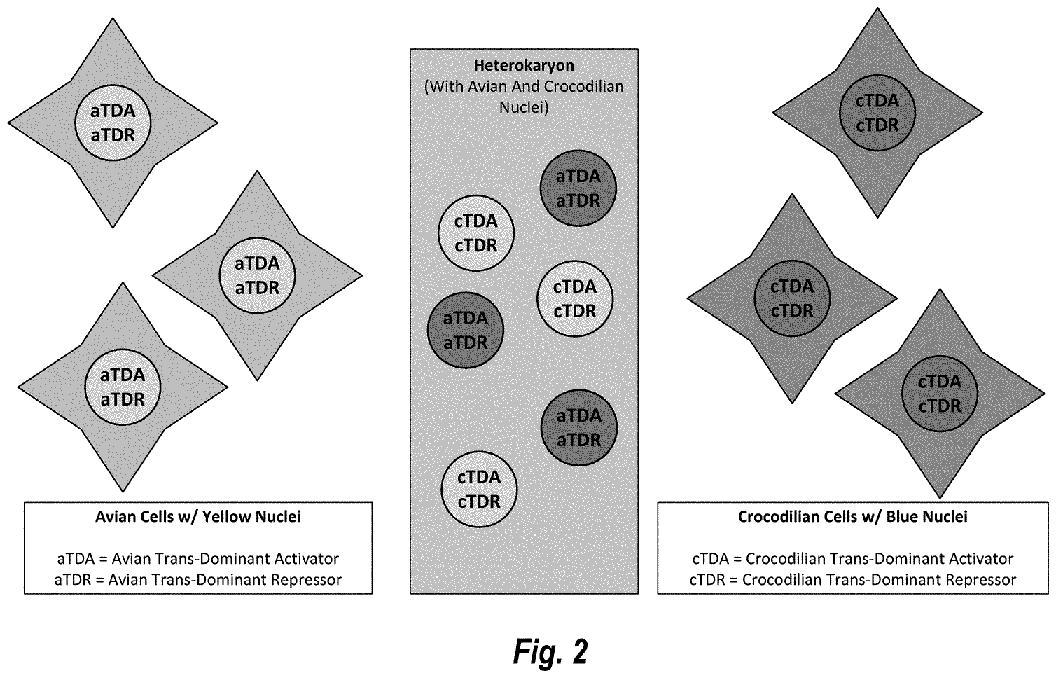

In a preferred second embodiment, cells from sibling extant taxa that comprise a common ancestral species may be fused to form multi-nucleated syncytia known as heterokaryons. Cell fusion may enable modulation (i.e. transcriptional activation or repression) of transcriptional regulatory mechanisms, wherein programs for such modulators active and originating from the nuclei of one species modulate transcription of the other species where such endogenous programs are inactive or non-existent. Such modulators are identified herein as trans-dominant. As shown in FIG. 2, examples of trans-dominant modulator effects include transcriptional activation by trans-dominant gene expression programs, that induce expression of genes from the common ancestor that were silenced over the course of evolution, and, reciprocally, transcriptional repression by trans-dominant gene expression programs, that silence expression of genes from the extant species that were activated over the course of evolution. Thus, biochemical features of extinct progenitors species may be resurrected in their extant descendant species by altering gene expression from the extant species genome. Such biochemical features may comprise proteins, adducts of proteins, polymeric sugars, post-translational modifications, metabolic intermediates, structural RNAs, steroidal compounds, and isoprenoid derivatives, such as lipids, fatty acids and steroids, whose presence or absence in extant species may be restored to functional levels comparable to those found in an extinct common ancestor. In certain embodiments, heterokaryons may comprise biochemical profiles common to the original extant taxa cells from which they were derived. Alternatively, heterokaryons may comprise a unique biochemical profile distinct from the original extant taxa cells from which they were derived. Heterokaryons that successfully express ancestral genes may then be subjected to the above cultivation procedures to generate cell-based products for consumption that recapitulate unique organoleptic and nutritional profiles from extinct, common ancestral species.

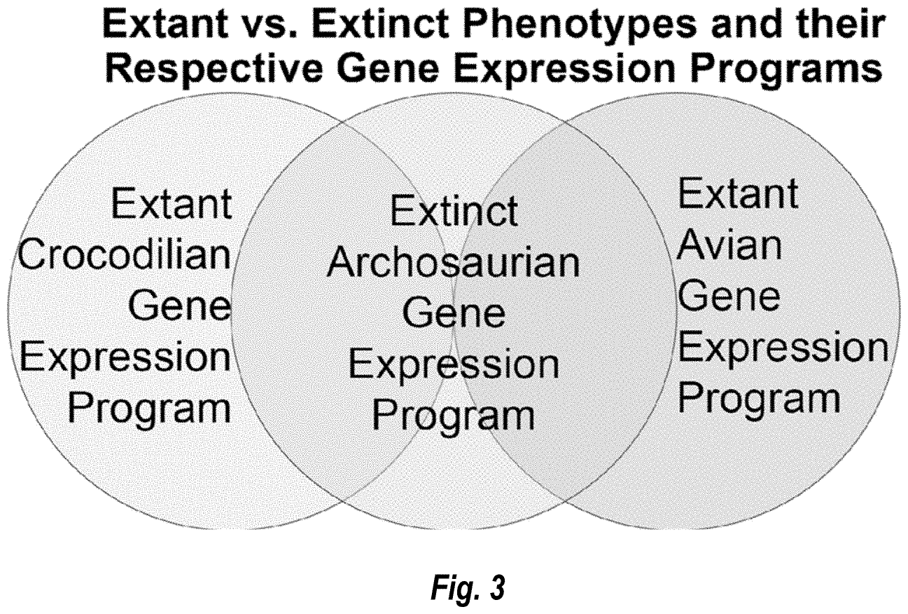

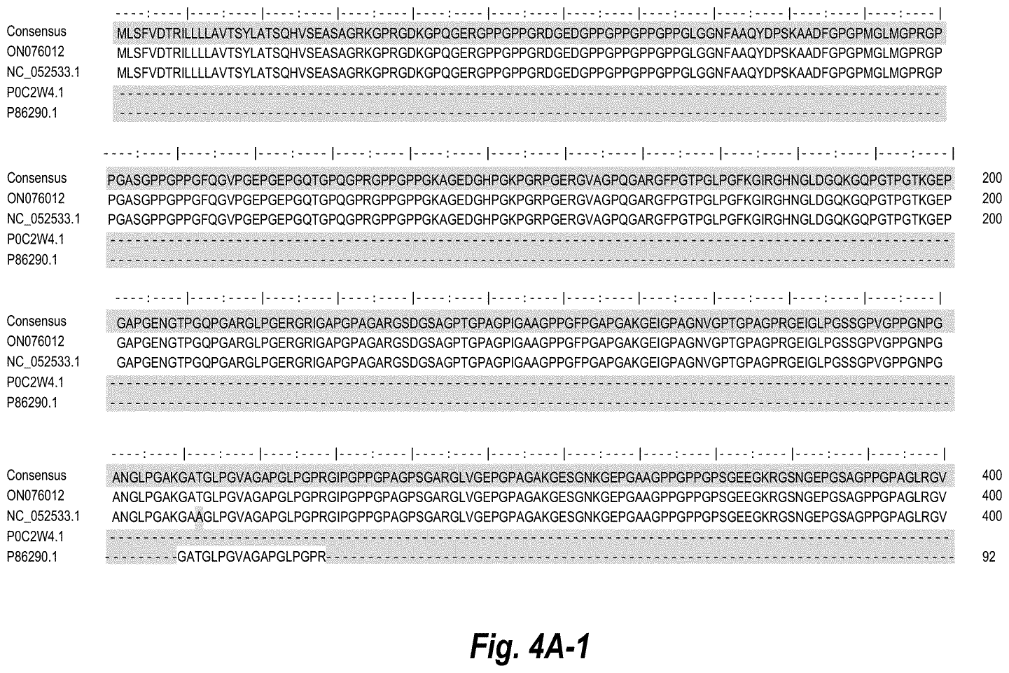

In a particular aspect, as shown in FIG. 3, cells from extant crocodilian and avian species may be fused together, activating a trans-dominant transcription program that induces expression of silenced genes from an archosaurian progenitor common to both species. The expressed genes may be (1) specific to the avian species and the archosaurian progenitor; (2) specific to the crocodilian species and the archosaurian progenitor; (3) specific to both the avian and crocodilian species as well as the archosaurian progenitor; or (4) specific only to the archosaurian progenitor. In alternative aspects, cells to be fused may come from at least two species categorized under one or more of the following metazoan extant metazoan categories: fish, mammals, amphibians, birds, reptiles, or invertebrates.

In a further embodiment, the endogenous transcriptional regulatory mechanisms involved in inducing expression of genes from the common ancestor may be spontaneous and not require genetic or epigenetic modification.

In a further embodiment, the native cell fusion mechanisms from two sibling taxa comprising the heterokaryon progenitor cells may be sufficient to enable cell fusion as a conduit for trans-dominant transcriptional modulation within the heterokaryon. In addition, native cell fusion mechanisms may be augmented, mediated or facilitated by one or more of the following: activation or overexpression of one or more of the following proteins: myomaker, myomixer, minion, myomeger, dysferlin, myoferlin, syncitin-1 or their homologs; application of polyethylene glycol; or application of an electric current.

In a further embodiment, trans-dominant transcriptional modulation between different nuclei within the heterokaryon originating from distinct species may be spontaneous and not require further alteration of the chromatin through the addition and or removal of epigenetic modifications such as DNA methylation, histone methylation and histone acetylation to convey or augment such trans-dominant transcriptional modulation.

In a further embodiment, trans-dominant transcriptional modulation between different nuclei within the heterokaryon originating from distinct species may be enabled or augmented by further alteration of the chromatin through the addition and or removal of epigenetic modifications such as DNA methylation, histone methylation and histone acetylation to convey or augment such trans-dominant transcriptional modulation upon the affected chromatin. Epigenetic modifiers may include one or more of the following factors: 5-azacytidine, 5-aza-2′-deoxycytidine, trichostatin A, valproic acid, butyrate, stilbenoid activators of sirtuin homologs such as 3,5,4′-trihydroxy-trans-stilbene, histone deacetylase enzymes, histone acetyltransferase enzymes, histone methyltransferase enzymes, histone demethylase enzymes and even ten-eleven translocation (TET) methylcytosine dioxygenase enzymes.

Although a minimum of two nuclei from two different species may be required to generate a heterokaryon in the application of this invention, the classifications of nuclei are, for the purposes of this invention, not limited to two. For example, cells from three or more species can be fused into a heterokaryon wherein a more comprehensive set of complimentary trans-dominant transcriptional modulators alone or in combination with target genetic sequences for transcriptional modulation may support a biochemical profile more closely resembling the targeted profile. Beyond having nuclei from two different species, the distinctions introduced by the third nuclei in such heterokaryons are not limited to species distinction. For example, the heterokaryon may consist of nuclei originating from myoblasts of species A, nuclei originating from myoblasts of species B, and nuclei originating from fibroblasts of species A. Wherein it is expected that the genetic sequences in the species A myoblasts and fibroblasts would substantially be the same, the trans-dominant transcriptional modulators expressed by these two cell lineages may differ, thereby adding further diversity to the transcriptional control mechanisms within the heterokaryon. In certain embodiments, the heterokaryon may represent one or more tissue lineages.

Particularly preferable embodiments of this invention include methods of synthesizing extinct muscle derived tissue, comprising: selecting an extinct genome comprising at least a portion of a genome of an extinct species; selecting a foundational genome comprising at least a portion of a genome of a non-extinct ancestor to the extinct species; identifying absent genes, the absent genes comprising genes present in the extinct genome and absent from the foundational genome; combining the foundational genome and the absent genes to generate a hybridized genome; expressing the absent genes of the hybridized genome in a growing cell mass; and preparing the grown cell mass for consumption as a cell-based meat product.

In alternative embodiments, the genes in the extinct genome may exhibit distinct expression from sister genes in the foundational genome and serve as replacements or substitutions for the sister genes of the foundational genome.

In further embodiments, the extinct species may be a mammoth and the non-extinct ancestor may be an elephant. Alternatively, the extinct species may be a dinosaur and the non-extinct ancestor may be a chicken.

The absent genes may comprise only genes that support muscle tissue growth, such as collagen or myosin genes. Alternatively, the absent genes may comprise actin, creatine kinase, tropomyosin, fibronectin, myoglobin, or keratin.

In some embodiments, the grown cell mass may comprise cells of the non-extinct ancestor transfected with the hybridized genome. In particular examples, the cells may be derived from chicken and the hybridized genome may be codon optimized for expression by cultured chicken cells.

In further embodiments, the foundational genes of the hybridized genome may be expressed in the growing cell mass. In addition, the absent genes may be overexpressed using a promoter. In certain embodiments, the extinct genome may comprise at least a portion of two or more extinct species.

Alternative embodiments may comprise a cell-based food product for consumption comprising extinct muscle derived tissue, the extinct muscle derived tissue comprising at least one gene from an extinct species and at least one gene from a non-extinct ancestor to the extinct species, wherein the at least one gene from the extinct species is absent from the genome of the non-extinct ancestor.

Alternatively, the at least one gene from the extinct species may be substituted for a sister gene from the non-extinct ancestor.

The above embodiments are by no means limiting and include any combination of extinct and foundational genes that fall within the present disclosure. This invention is further illustrated by the following additional examples that should not be construed as limiting. Those of skill in the art should, in light of the present disclosure, appreciate that many changes can be made to the specific embodiments which are disclosed and still obtain a like or similar result without departing from the spirit and scope of the invention.

All of the claims in the claim listing are herein incorporated by reference into the specification in their entireties as additional embodiments.

### EXAMPLES

### Example 1: Prediction of Repressed Gene Expression Profiles and Protein Expression Profiles in Avian-Crocodilian Homokaryons and Heterokaryons

Primary Isolation of Satellite Cells from Avian and Crocodilian Species.

Cells were isolated from a juvenile chicken (Gallus gallus) and a juvenile American alligator (Alligator mississippiensis) by the following procedure. Tissues samples were aseptically obtained from the pectoralis muscle group from sub-adult specimens of each species using methodologies generally known in the art to support the isolation of satellite cells from skeletal muscle tissues originating from a plurality of vertebrate species. The obtained tissue was enzymatically dissociated to release satellite cells from the skeletal muscle explants. Tissue debris was separated from the isolated cells by filtration through a fine mesh. Cells were separated from the enzymatic digest solution by centrifugation and resuspended in a growth medium consisting of a Dulbecco's Modified Eagle Medium containing a bicarbonate buffering system and supplemented with glutamine, ungulate serum, chick embryo extract and an antibiotic/antimycotic solution.

### Cultivation of Crocodilian and Avian Myoblasts

Satellite cells isolated from the avian and crocodilian tissues were seeded onto tissue-culture grade polystyrene culture dishes in the growth medium onto surfaces previously coated with gelatin. Culture dishes seeded with crocodilian cells were incubated at temperatures ranging from 86-93° C., whereas cells isolated from avian tissue were incubated at 37° C. From this point, proliferating progeny of the isolated satellite cells are identified as myoblasts. All cultures were incubated under 5% atmospheric carbon dioxide culture conditions. The temperature condition supporting the most robust cell outgrowth of the crocodilian myoblasts was first used to expand the population of crocodilian myoblasts. Next, cells were passaged from this condition to otherwise equivalent conditions ranging in temperature from the starting condition supporting the most robust cell growth to 37° C. The temperature closest to 37° C. capable of supporting a cell population crocodilian cell doubling rate below forty-eight hours was selected to co-culture avian and crocodilian myoblasts.

### Co-Culture and Fusion of Crocodilian and Avian Myoblasts

Avian cultures and selected crocodilian cultures were enzymatically dissociated and seeded onto gelatin-coated tissue culture grade plastic dishes in growth medium at 15,000 cells/cm² for each cell population, for a total seeding density of 30,000 cells/cm². The co-culture was returned to the incubator setting for the selected crocodilian cultures. Once this co-culture reached approximately 80% confluence, ungulate serum in the growth medium was reduced and ungulate insulin-like growth-factor was added to support myoblast fusion within the co-culture and formation of myotube heterokaryons consisting of both crocodilian and avian nuclei. Incubation of the co-cultures was continued until myotubes in the culture dish reached their maximum size, at which point cellular biomass is collected for processing for downstream analytics or as a food ingredient or product.

Identification of Repressed Avian and Crocodilian Gene Expression Programs Activated in Crocodilian-Avian Cell Heterokaryons

The defining aspects of this innovation include (1) methods for activating expression of repressed gene expression programs in Avian and Crocodilian species as described in an exemplary manner in this example, and (2) protein and lipid profile constituency subsequent to activated gene expression profiles in Avian and Crocodilian heterokaryons not found in either the Avian or Crocodilian gene expression programs of the contributing myoblasts from the respective constituent species represented within the co-cultures previously described within this example.

In a separate experiment, avian or crocodilian myoblasts were seeded and differentiated into myotubes as described in the procedures for establishing avian-crocodilian heterokaryons, except that both avian and crocodilian myoblasts were seeded at 30,000 cells per cm² and directed to fuse and form myotubes as species-exclusive monocultures, resulting in homokaryons. Once the homokaryon myotubes reached their maximum size, their cellular biomass was collected and processed for downstream analytics.

Relevant downstream analytics for determining the gene expression profiles and predicting the protein expression profiles of the heterokaryon and homokaryon cultures include RNA-seq, and variations of mass spectroscopy such as LC-MS to determine lipid profiles. Activated gene expression profiles present in heterokaryon cultures but repressed in either homokaryon cultures will be determined by subtracting orthologous gene expression common to both homo- and heterokaryon cultures from heterokaryon gene expression profiles. The remaining heterokaryon gene expression profile will be classified and heterokaryon-transactivated gene expression predictive of gene-expression programs expressed in the archosaurian ancestors of avian and crocodilian species no longer expressed in chickens and alligators. Expression of enzymatic proteins of lipid biosynthetic pathways arising from heterokaryon-transactivated gene expression in conjunction with metabolites are predicted to result in the biosynthesis of cellular lipid molecules found neither in the crocodilian nor the avian lipid profiles displayed within their respective homokaryons. These lipid profiles define the major component of the flavor profile of meat in a species-specific manner in foods prepared from tissues of the respective species. Therefore, the organoleptic phenotype of heterokaryon-activated lipid biosynthetic pathway profiles is predicted to impart within foods prepared from the respective heterokaryon biomass authentic flavor notes of the extinct archosaurian ancestors of avian and crocodilian species no longer present in alligator or chicken muscle.

### Example 2: Expression of Dinosaur Collagen 1A1 and 1A2 in Cultured Chicken Cells

This experiment proposes to introduce ancient DNA coding for Tyrannosaurus Rex and Brachylophosaurus canadensis collagen into cultured chicken cells for the purpose of food product development. Specifically, extinct collagen genes from extinct Tyrannosaurus Rex and Brachylophosaurus canadensis species were mapped against foundational genes from chickens, a non-extinct ancestor to Tyrannosaurus Rex and Brachylophosaurus canadensis, to identify collagen genes present in the extinct dinosaur species and absent in chickens. The dinosaur-specific collagen genes were then combined with the chicken genes and expressed in a growing culture of chicken cells. The mass of dinosaur collagen-expressing cultured chicken cells was grown to generate a cell-based meat product for consumption.

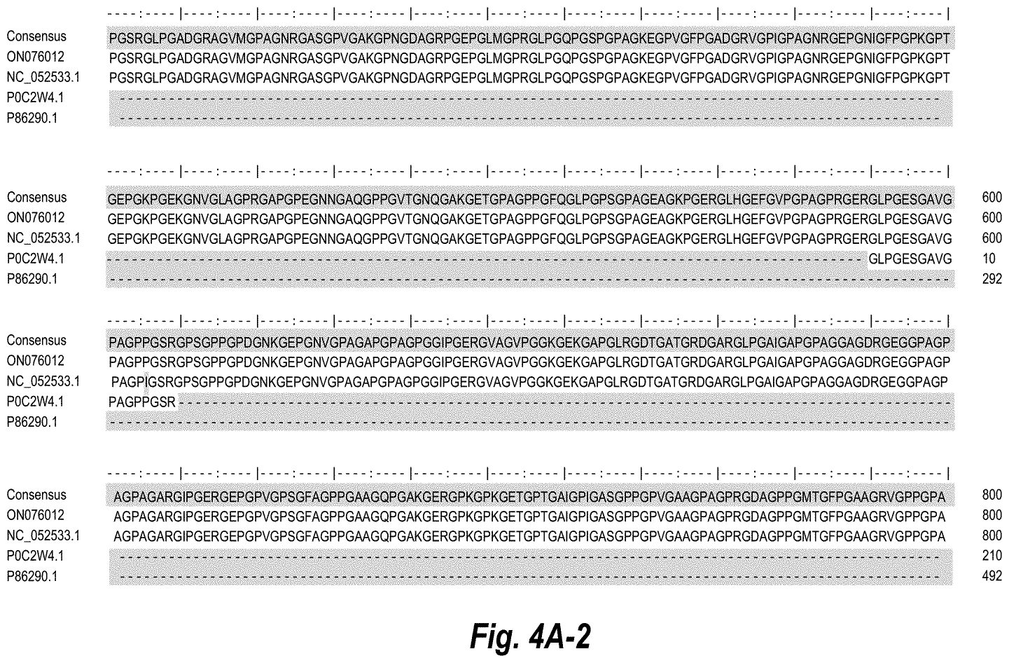

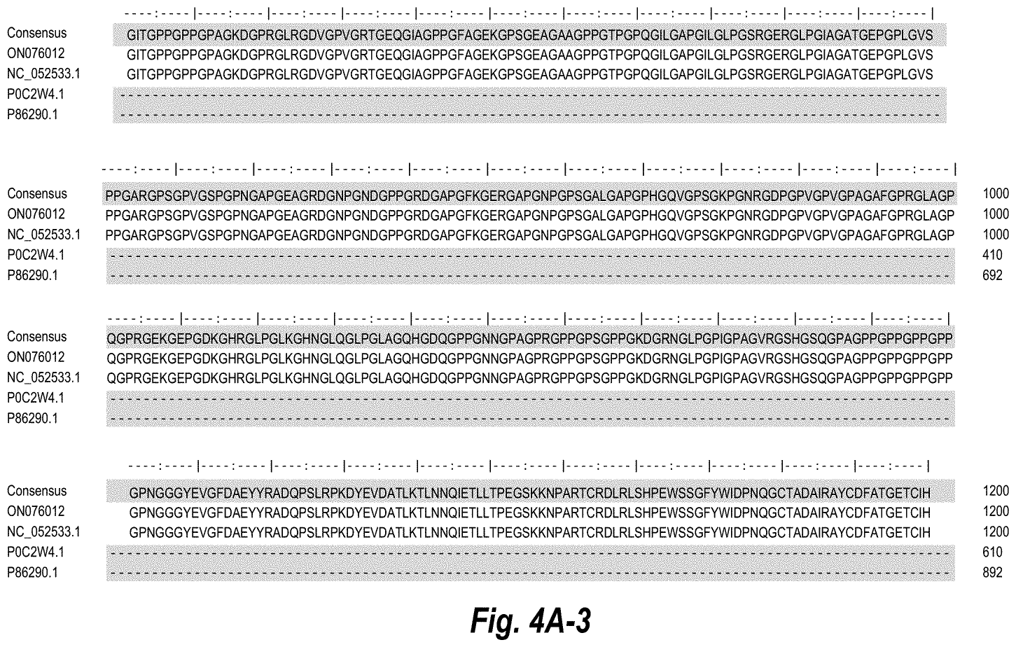

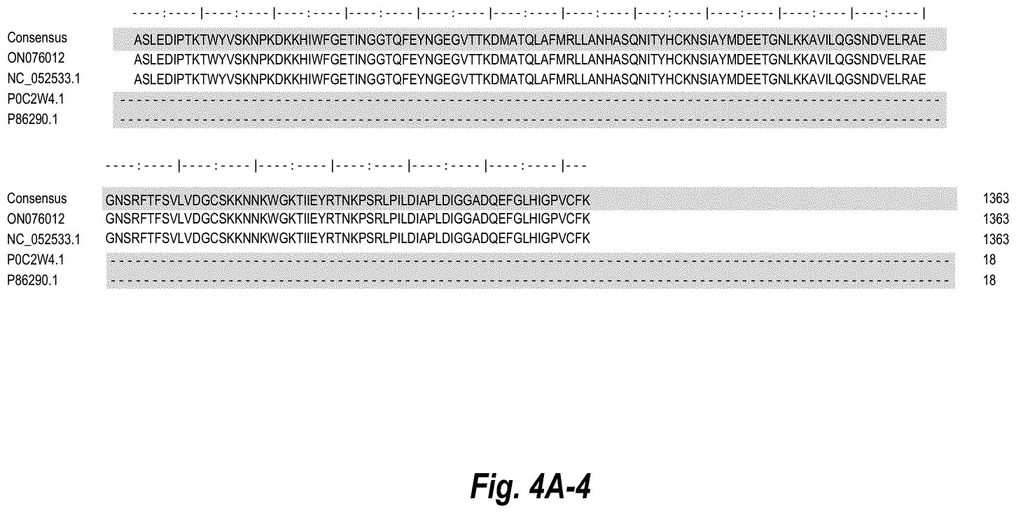

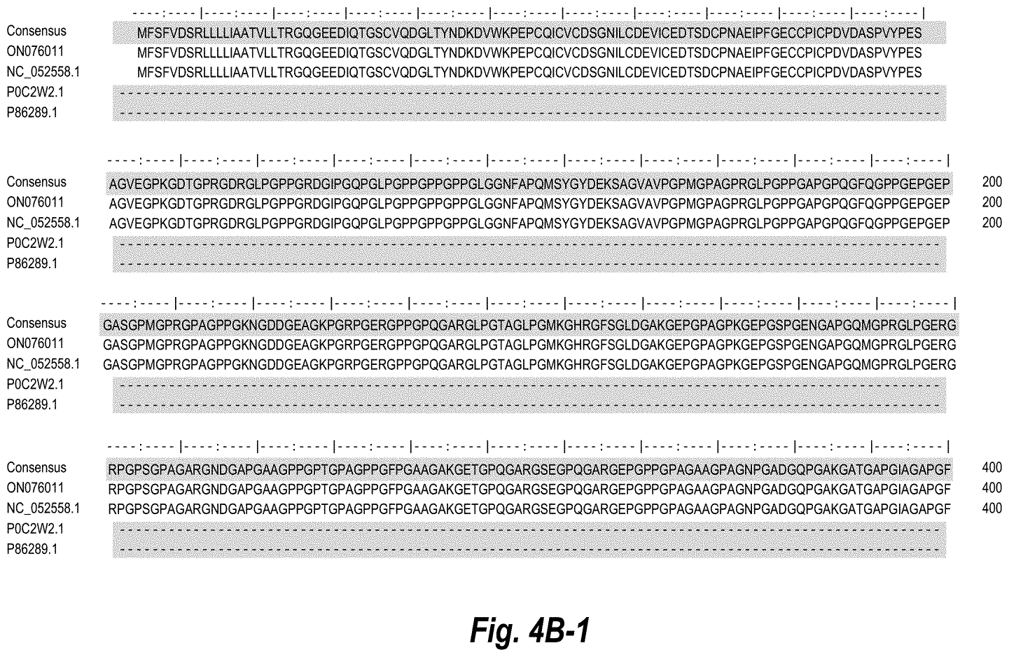

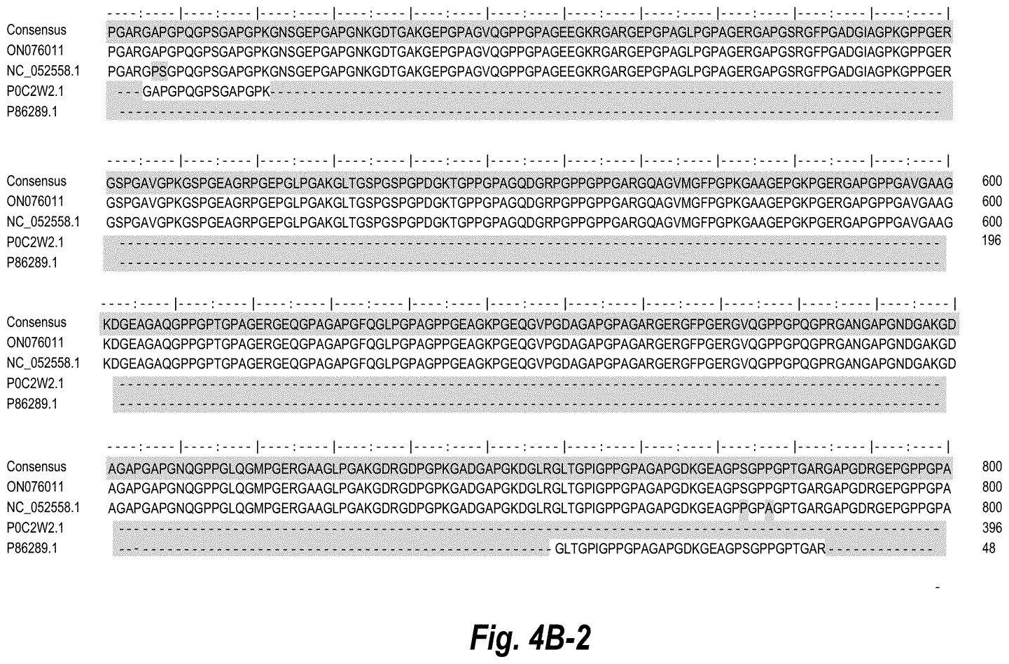

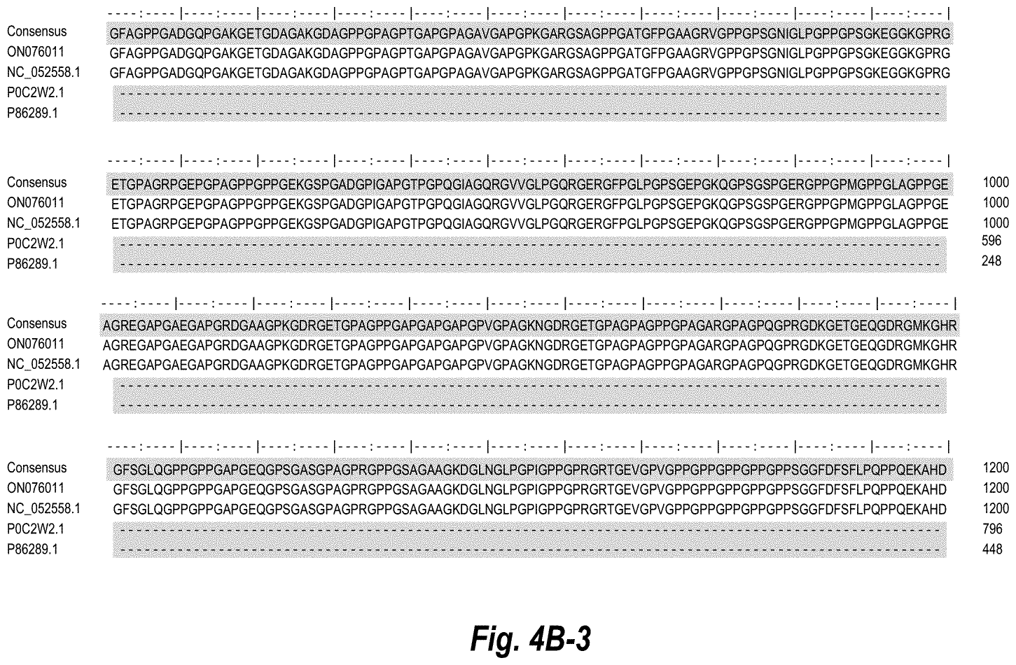

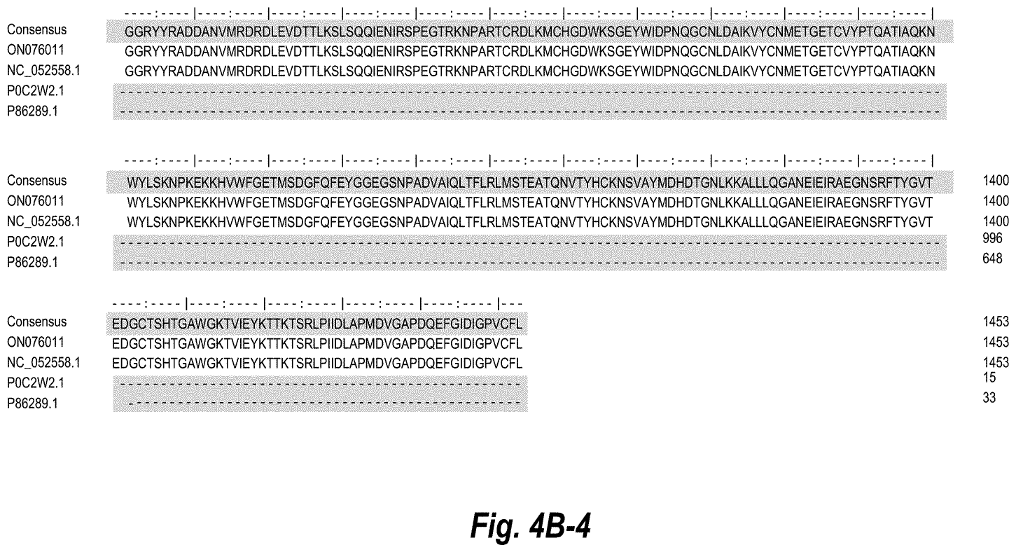

As shown in FIGS. 4A-1-4A-4 and 4B-1-4B-4, Tyrannosaurus Rex and Brachylophosaurus canadensis collagen I, alpha I (COL 1A1) and collagen I, alpha 2 (COL 1A2) sequences were mapped to reference chicken COL 1A1 and COL 1A2 proteins to identify any amino acid sequence divergences that exist between the species. Relative to chicken COL 1A1, amino acid substitutions P-406-A and S-407-P were found to be unique to Tyrannosaurus Rex, and amino acid substitutions P-776-S and A-779-P were found to be unique to Brachylophosaurus canadensis. Relative to chicken COL 1A2, amino acid substitution I-605-P was found to be unique to Tyrannosaurus Rex, and amino acid substitution A-311-T was found to be unique to Brachylophosaurus canadensis.

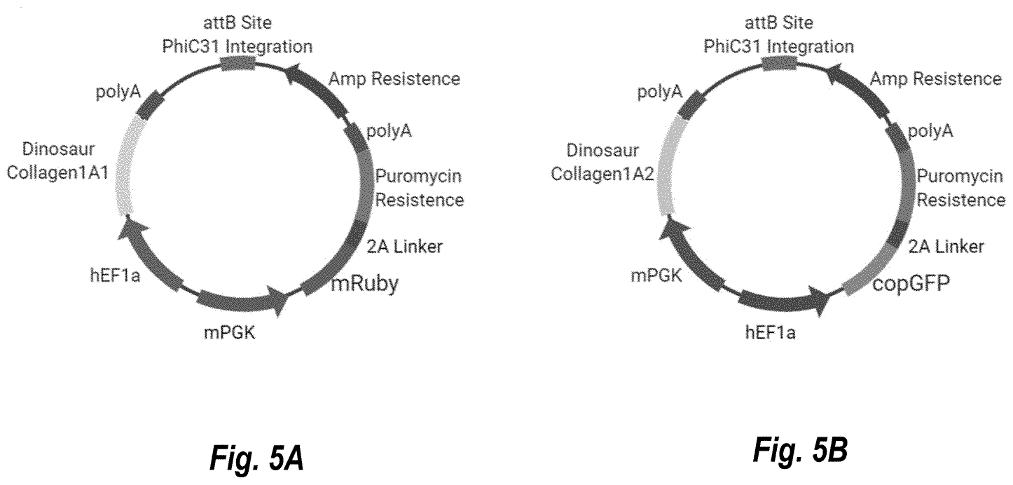

As shown in FIGS. 5A and 5B, the dinosaur-specific COL1A1 (P-406-A, S-407-P, P-776-S and A-779-P) amino acid substitutions were inserted into the chicken COL1A1 reference protein sequence, chicken codon optimized, and designated Dinosaur COL1A1. The dinosaur-specific COL1A2 (I-605-P, A-311-T) amino acid substitutions were inserted into the chicken COL1A2 reference protein sequence, chicken codon optimized, and designated Dinosaur COL1A2. The Dinosaur COL1A1 gene sequence was chicken codon optimized and cloned into a PhiC31 vector under the control of an EF1a promoter. The Dinosaur COL1A2 gene sequence was codon optimized and cloned into a PhiC31 vector under control of a PGK promoter. The Dinosaur COL1A1 and 1A2 vectors were used to develop stable expression in cultured chicken cells for cultured meat while not reducing expression of any chicken-specific genes.

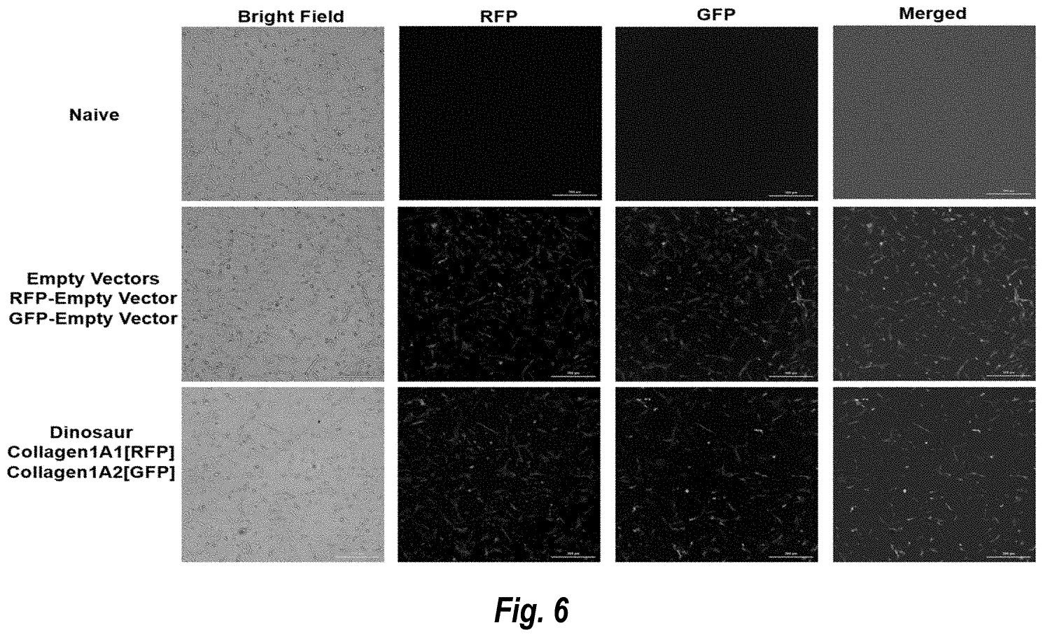

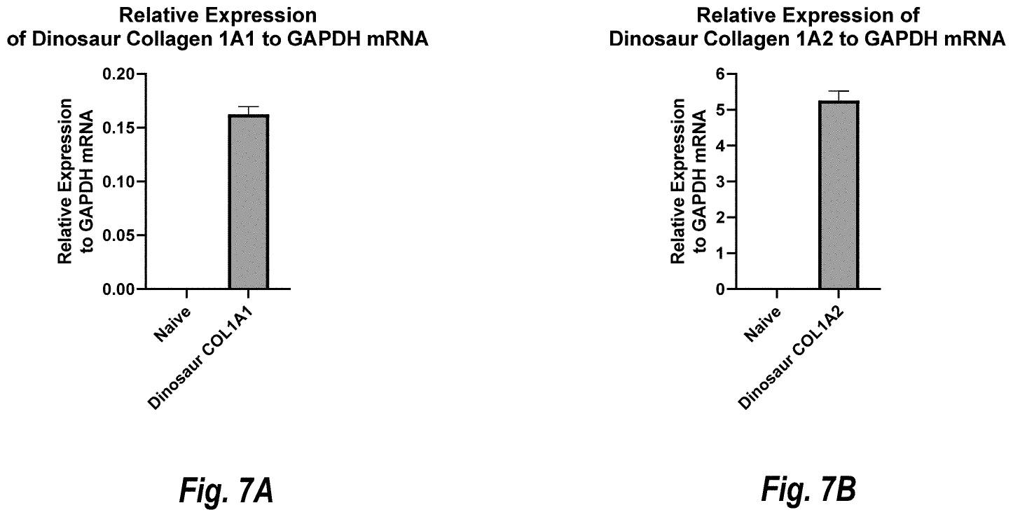

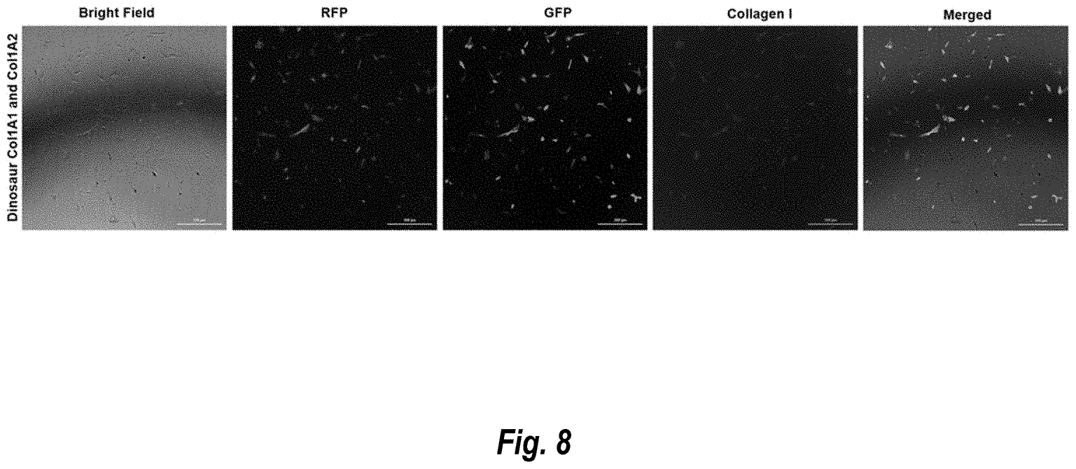

As shown in FIG. 6, cultured chicken cells were transfected with the Dinosaur COL1A1 and 1A2 vectors. Dinosaur collagen 1A1 and 1A2 gene vectors or control empty vectors were transfected with integrase into cultured chicken cells. A naïve sample served as an additional control. As demonstrated by red fluorescent and green fluorescent staining in both the empty vector and dinosaur-specific panels, transfection of cultured chicken cells was successful. FIGS. 7A and 7B serve as proof of concept that this transfection included dinosaur-specific collagen 1A1 and 1A2. Dinosaur collagen 1A1 and 1A2 levels were clearly significantly enhanced relative to naïve controls, indicating that genes for the specific dinosaur proteins were successfully inserted into cultured chicken cells and expressed. And FIG. 8 demonstrates that expression of the Dinosaur collagen 1A1 and 1A2 correlates with expression of collagen, illustrating that the dinosaur-specific collagen proteins map to chicken-specific collagen. These findings demonstrate that dinosaur-specific collagen proteins can be successfully expressed in cultured chicken cells.

### Example 3: Expression of Mammoth Myosin Heavy Chain MYH13 in Cultured Chicken Cells

This experiment proposes to introduce ancient DNA coding for Mammoth-specific myosin heavy chain protein into cultured chicken cells for the purpose of food product development. Specifically, extinct myosin genes from an extinct Woolly Mammoth species were mapped against foundational genes from non-extinct elephants, a non-extinct ancestor to Woolly Mammoth, to identify myosin genes in Woolly Mammoth that exhibit differential expression than their sister counterpart in elephants. The differentially expressed Woolly Mammoth-specific myosin genes were then substituted for the sister elephant genes and expressed in a growing culture of chicken cells. The mass of Woolly Mammoth myosin-expressing cultured chicken cells was grown to generate a cell-based meat product for consumption.

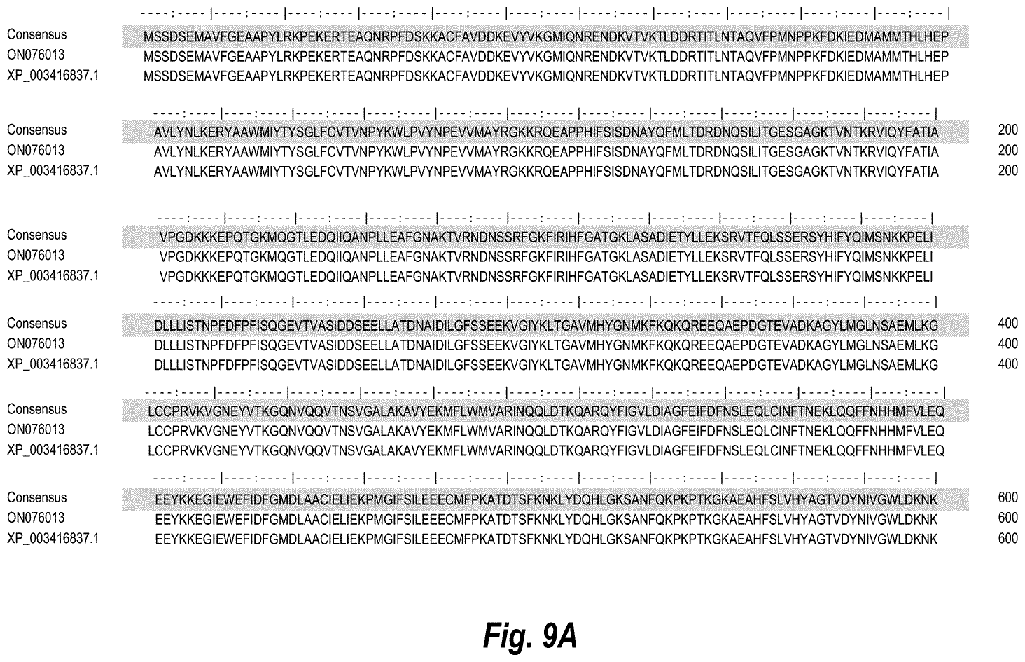

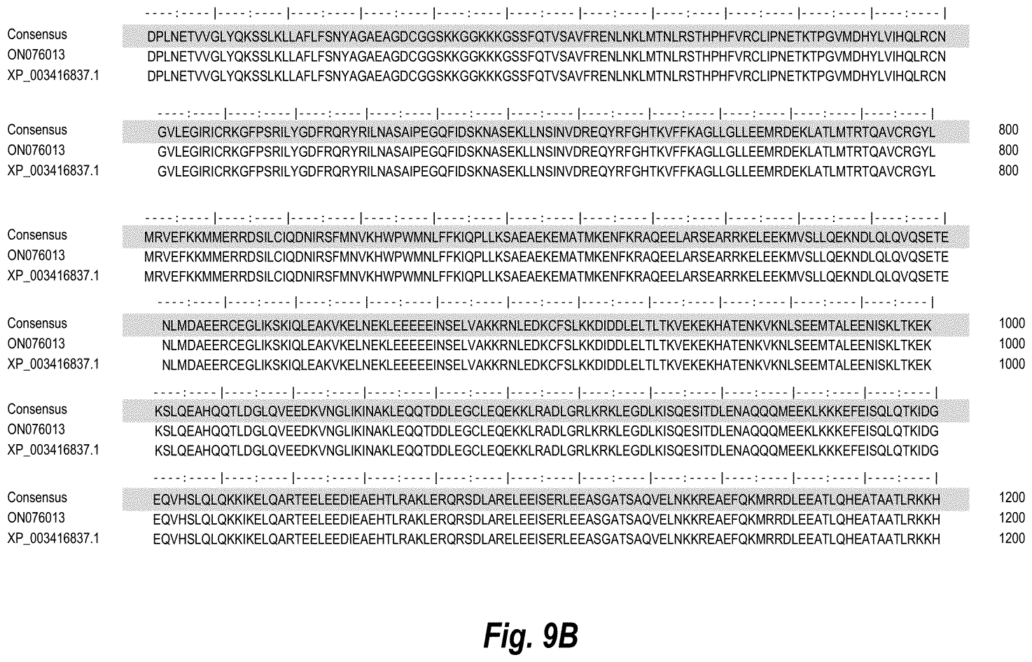

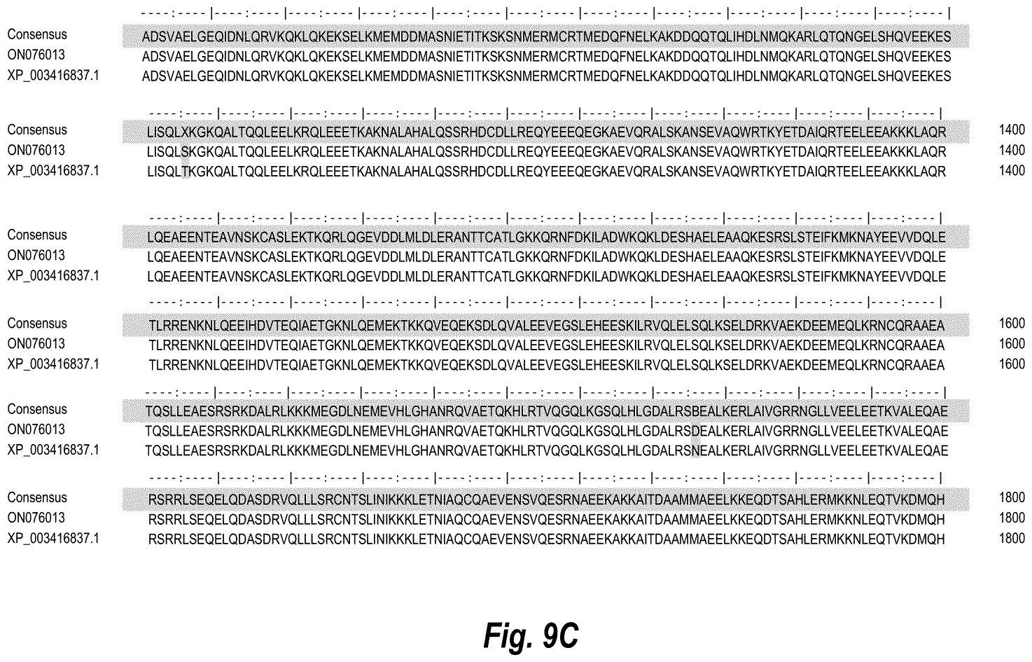

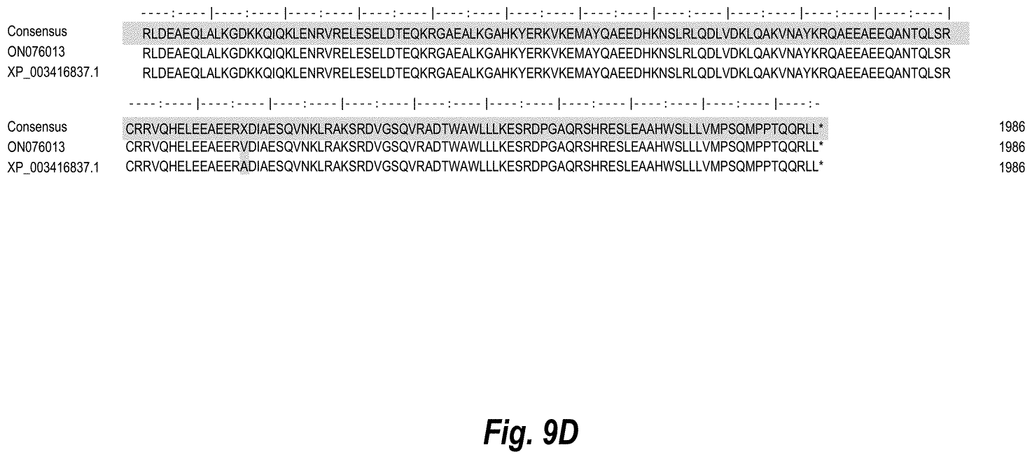

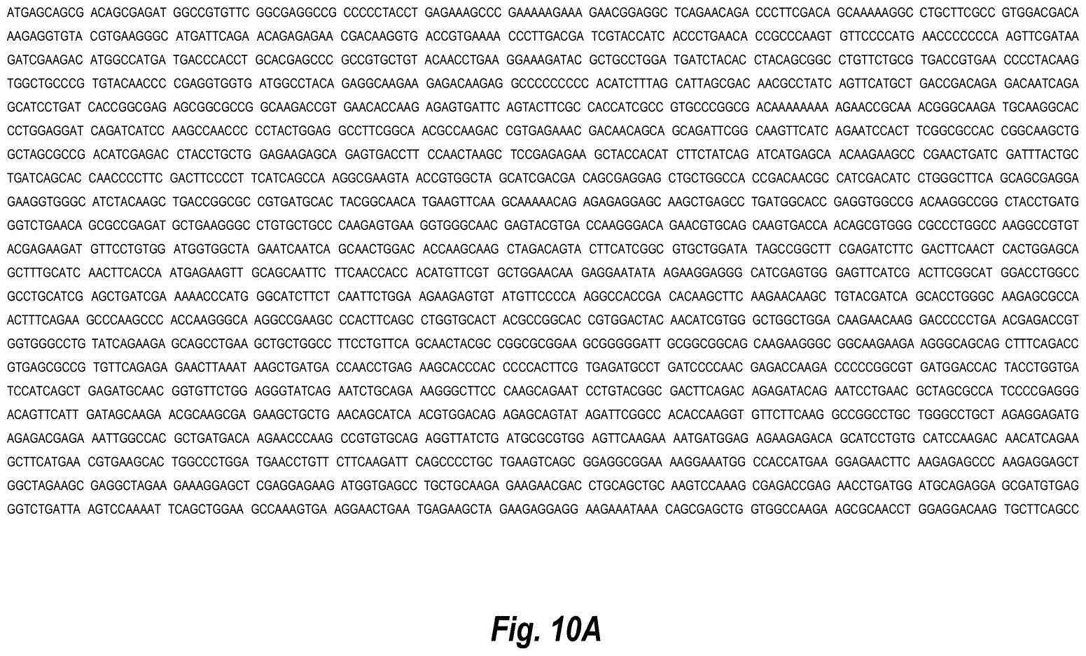

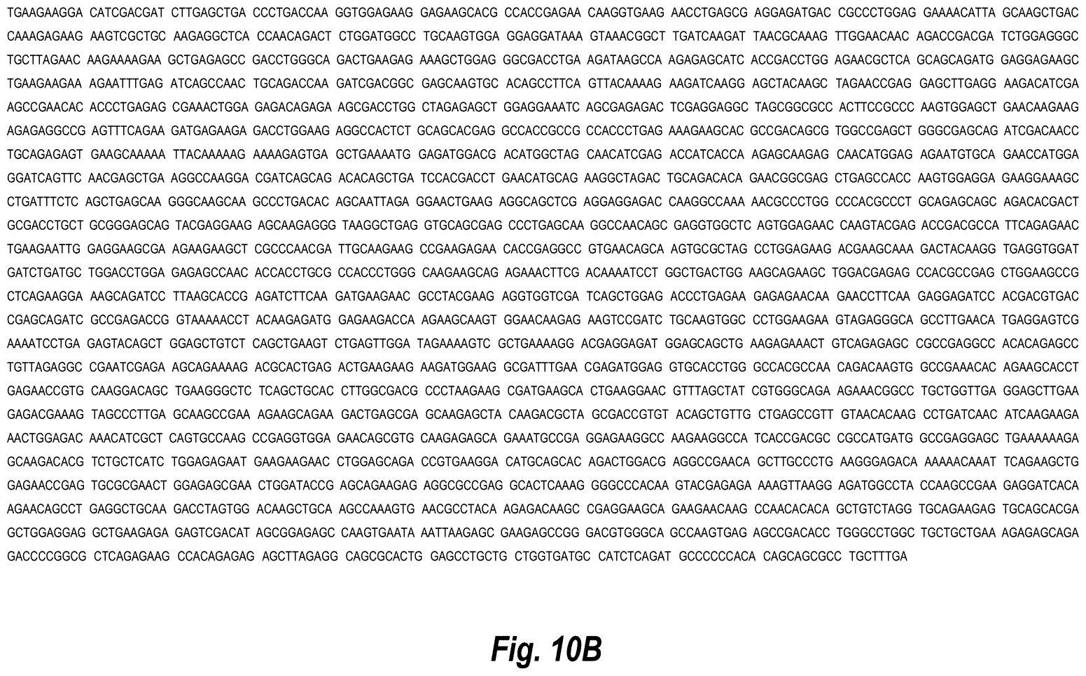

Mammoth and elephant genomes were analyzed for protein coding non-synonymous single nucleotide polymorphisms (SNPs) that only exist in Woolly Mammoth Myosin genes. Elephant genomes examined included African elephant (L_africana_B, L_africana_C, LoxAfr 3.0), Asian elephant (E_maximus_D, E_maximus_E), Forest Elephant (L_cyclotis_A, L_cyclotis_F), Straight tusked elephant (P_antiquus_N, P_antiquus_O). Mammoth genomes included in the analysis were Woolly Mammoth (Wrangle, Oimyakon, M_primigenius_G, M_primigenius_H, M_primigenius_S, M_primigenius_V) and Columbian Mammoth (M_columbi_U). As shown in FIGS. 9A-9D, mammoth specific protein coding SNPs were identified in myosin heavy chain gene MYH13 to include T-1306-S, N-1668-D, A-1914-V. These amino acid substitutions did not exist in any elephant genomes analyzed. The identified amino acid substitutions were applied to the Loxodonta africana MYH13 sequence. As shown in FIGS. 10A-10B, the mammoth myosin heavy chain gene (MYH13) containing amino acids 1914-V, 1668-D, and 1306-S was created by mapping the amino acid substitutions to the Loxodonta africana reference genome. The nucleotide sequence was chicken codon optimized for expression in cultured chicken cells.

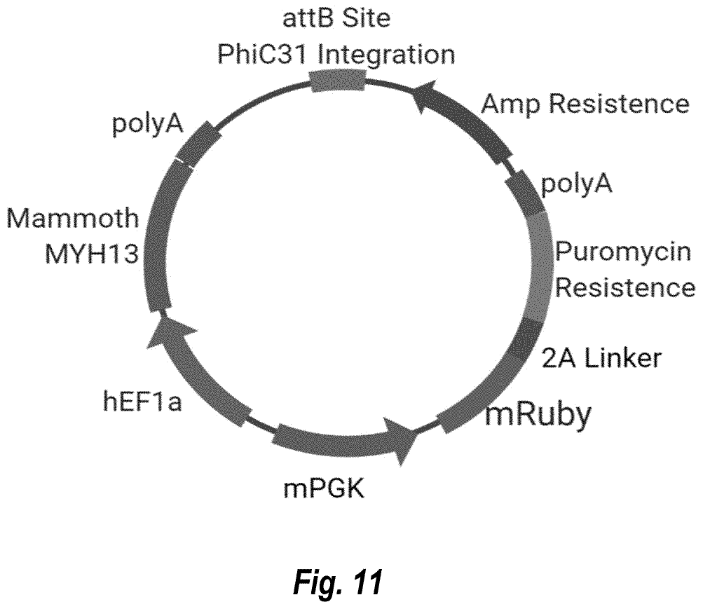

As shown in FIG. 11, the mammoth-specific MYH13 containing 1914-V, 1668-D, and 1306-S amino acids was encoded in the Loxodonta africana MYH13 reference sequence. The nucleotide sequence was chicken codon optimized and cloned into a PhiC31-RFP vector for stable expression in cultured chicken cells developed for cultured meat.

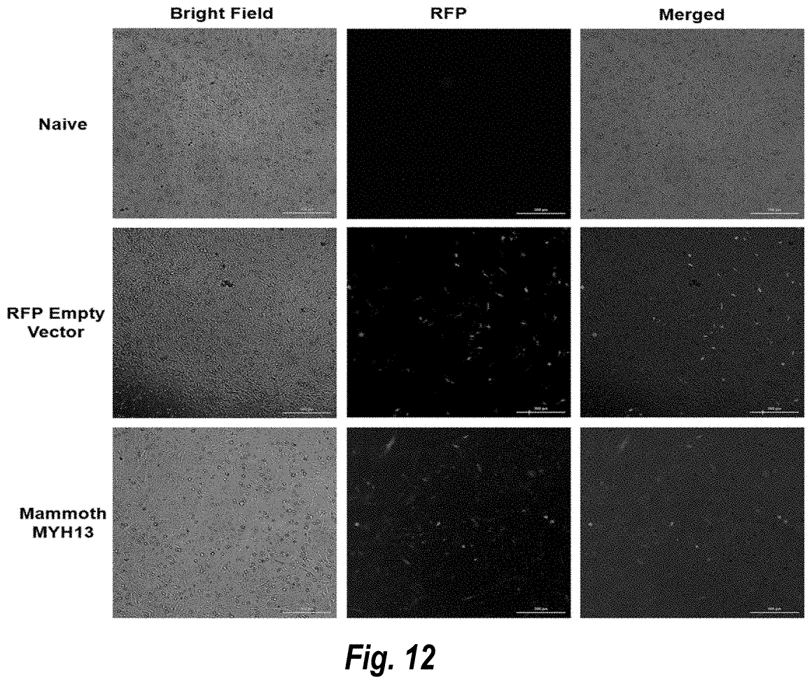

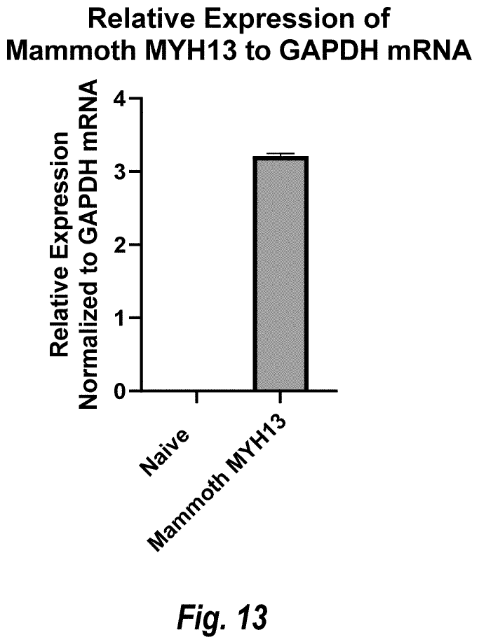

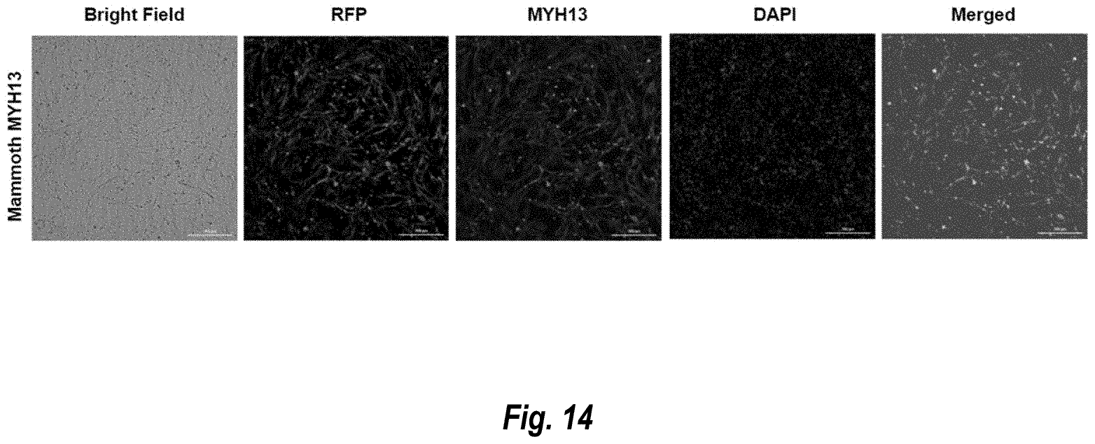

As shown in FIG. 12, cultured chicken cells were transfected with the Mammoth MYH13 gene vector. A Mammoth MYH13 gene gene vector or control empty vector were transfected with integrase into cultured chicken cells. A naïve sample served as an additional control. As demonstrated by red fluorescent staining in both the empty vector and mammoth-specific panels, transfection of cultured chicken cells was successful. FIG. 13 serves as proof of concept that this transfection included mammoth-specific MYH13. Mammoth MYH13 levels were clearly significantly enhanced relative to a naïve control, indicating that the gene for the specific mammoth protein was successfully inserted into cultured chicken cells and expressed. And FIG. 14 demonstrates that expression of the Mammoth MYH13 correlates with expression of myosin, illustrating that the mammoth-specific myosin proteins map to chicken-specific myosin. These findings demonstrate that mammoth-specific myosin proteins can be successfully expressed in cultured chicken cells.

### REFERENCES

All references referred to above are incorporated herein by reference in their entireties.

What is claimed is:

1. A method of synthesizing extinct muscle derived tissue, comprising:
selecting an extinct genome comprising at least a portion of a genome of an extinct species;
selecting a foundational genome comprising at least a portion of a genome of a non-extinct ancestor to the extinct species;
identifying at least one absent gene, the at least one absent gene comprising genes present in the extinct genome and absent from the foundational genome;
combining the foundational genome and the at least one absent gene to generate a hybridized genome;
expressing the at least one absent gene of the hybridized genome in a growing cell mass; and
preparing the grown cell mass for consumption as a cell-based meat product.

2. The method of claim 1, wherein the extinct species is a mammoth and the non-extinct ancestor is an elephant.

3. The method of claim 1, wherein the extinct species is a dinosaur and the non-extinct ancestor is a chicken.

4. The method of claim 1, wherein the at least one absent gene comprises only genes that support muscle tissue growth.

5. The method of claim 3, wherein the at least one absent gene comprises collagen genes.

6. The method of claim 2, wherein the at least one absent gene comprises myosin genes.

7. The method of claim 1, wherein the at least one absent gene comprises actin, creatine kinase, tropomyosin, fibronectin, myoglobin, or keratin.

8. The method of claim 1, wherein the grown cell mass comprises cells of the non-extinct ancestor transfected with the hybridized genome.

9. The method of claim 1, wherein the grown cell mass comprises cells derived from chicken transfected with the hybridized genome.

10. The method of claim 9, wherein the hybridized genome is codon optimized for expression by cultured chicken cells.

11. The method of claim 1, further comprising expressing the foundational genes of the hybridized genome in the growing cell mass.

12. The method of claim 1, wherein the at least one absent gene is overexpressed using a promoter.

13. The method of claim 1, wherein the extinct genome is comprised of at least a portion of two or more extinct species.

14. A method of synthesizing extinct muscle derived tissue, comprising:
selecting an extinct genome comprising at least a portion of a genome of an extinct species;
selecting a foundational genome comprising at least a portion of a genome of a non-extinct ancestor to the extinct species;
identifying at least one gene present in the extinct genome that exhibits distinct expression from a sister gene in the foundational genome;
substituting the sister gene of the foundational genome with the at least one extinct genome-specific gene to generate a hybridized genome;
expressing the at least one extinct genome-specific gene of the hybridized genome in a growing cell mass; and
preparing the grown cell mass for consumption.

15. The method of claim 14, wherein the extinct species is a mammoth and the non-extinct ancestor is an elephant.

16. The method of claim 14, wherein the extinct species is a dinosaur and the non-extinct ancestor is a chicken.

17. The method of claim 14, wherein the sister gene comprises only genes that support muscle tissue growth.

18. The method of claim 16, wherein the sister gene comprises collagen genes.

19. The method of claim 15, wherein the sister gene comprises myosin genes.

20. The method of claim 14, wherein the sister gene comprises actin, creatine kinase, tropomyosin, fibronectin, myoglobin, or keratin.

21. The method of claim 14, wherein the grown cell mass comprises cells of the non-extinct ancestor transfected with the hybridized genome.

22. The method of claim 14, wherein the grown cell mass comprises cells derived from chicken transfected with the hybridized genome.

23. The method of claim 22, wherein the hybridized genome is codon optimized for expression by cultured chicken cells.

24. The method of claim 14, further comprising expressing the foundational genes of the hybridized genome in the growing cell mass.

25. The method of claim 14, wherein the at least one absent gene is overexpressed using a promoter.

26. The method of claim 14, wherein the extinct genome is comprised of at least a portion of two or more extinct species.

27. A cell-based food product for consumption, comprising extinct muscle derived tissue, the extinct muscle derived tissue comprising at least one gene from an extinct species and at least one gene from a non-extinct ancestor to the extinct species; wherein the at least one gene from the extinct species is absent from the genome of the non-extinct ancestor.

28. A cell-based food product for consumption, comprising an extinct muscle derived tissue, the extinct muscle derived tissue comprising at least one gene from an extinct species and at least one gene from a non-extinct ancestor to the extinct species; wherein the at least one gene from the extinct species is substituted for a sister gene from the non-extinct ancestor.
